(19) United States
(12) Patent Application Publication
(10) Pub. No.: US 2025/0354980 A1
(43) Pub. Date: Nov. 20, 2025

(54) LYMPHOCYTE STIMULATION ASSAY TO QUANTIFY IMMUNE RESPONSES

(71) Applicant: The Regents of the University of California, Oakland, CA (US)

(72) Inventors: Yusaku Michael Shino, Los Angeles, CA (US); John A. Belperio, Los Angeles, CA (US)

(73) Assignee: The Regents of the University of California, Oakland, CA (US)

(21) Appl. No.: 18/867,634

(22) PCT Filed: Jun. 9, 2023
(86) PCT No.: PCT/US23/68219

§ 371 Date: Nov. 20, 2024

Related U.S. Application Data
(60) Provisional application No. 63/350,797, filed on Jun. 9, 2022.

(51) Int. Cl. G01N 33/50

(52) U.S. Cl. CPC G01N 33/5091; G01N 33/5023; G01N 33/5047

(57) ABSTRACT

Immune responses in mammals are mediated by a complex interaction between peripheral blood cells called leukocytes and signaling molecules called cytokines. The present invention provides assay systems for investigative, diagnostic, and therapeutic use assessing immune response. These and other aspects of the invention yield powerful tools and methods for characterizing patient immune responses in various pathological conditions, as well as for assessing immune modulatory agents.

### CROSS-REFERENCE TO RELATED APPLICATIONS

This application claims the benefit under 35 U.S.C. Section 119(e) of co-pending and commonly-assigned U.S. Provisional Patent Application Ser. No. 63/350,797, filed on Jun. 9, 2022, and entitled “A NOVEL LYMPHOCYTE STIMULATION ASSAY TO QUANTIFY IMMUNE RESPONSES” which application is incorporated by reference herein.

### TECHNICAL FIELD

Embodiments of the disclosure concern at least the fields of immunology and medicine.

### BACKGROUND OF THE INVENTION

Immune responses in mammals are mediated by a complex interaction between peripheral blood cells called leukocytes and signaling molecules called cytokines. Leukocytes arise from hematopoietic stem cells and undergo differentiation to cell types including monocytes, macrophages, eosinophils, basophils, neutrophils, B lymphocytes (B-cells), T lymphocytes (T-cells), and NK cells. Cytokines are a large and diverse group of proteins released by a broad range of cells including leukocytes as signaling molecules. Cytokines include chemokines, interferons, interleukins, lymphokines and tumor necrosis factors and regulate multiple functions including upregulation/downregulation of genes and transcriptions factors, cell recruitment, differentiation, proliferation and regulation.

Immune responses can be further categorized as innate and adaptive. The innate immune response involving macrophages, eosinophils, basophils, neutrophils and NK cells is evolutionary older and quick to respond, but non-specific in terms of antigens (targets of immune recognition). The adaptive immune response involving B and T-cells cells is slower to respond to new antigens but is able to form memory cells and produce a rapid and potent response when the antigen is encountered again. B-cell, by producing antibodies that bind to specific molecules called antigens, are responsible for humoral immunity. T-cells have antigen-specific T-cell receptors (TCRs) on their cell surface and are in part responsible for cellular immunity. There are different types of T-cells including: CD4+ “helper”, CD8+ “cytotoxic”, regulatory T-cells (Tregs), each performing its unique functions. Activation of T-cells occurs when an antigen-presenting cell (APC) bearing cognate antigen (Ag) in a major histocompatibility complex (MHC) protein interacts with a TCR specific for that antigen.

Immune responses are also determined by the stimulating antigens. For example, invading bacteria can initiate both an innate and adaptive immune response. During an innate response, phagocytes including macrophages and dendritic cells, can engulf and kill the invading bacteria, while neutrophils release an assortment of antimicrobial proteins. The adaptive immune response against invading bacteria can be initiated by the formation of complexes between an antigen presenting cell MHC Class II receptor on an antigen presenting cell and bacterial antigen, which trigger activation of CD4⁺ T cells. By contrast, immune responses against some viral infections can be initiated by the formation of MHC Class I/viral antigen complexes and subsequent activation of CD8⁺ cells. B and T-cells can be either naïve meaning that they have never encountered the antigen before, or memory cells meaning that they were previously exposed to the antigen. Although the adaptive immune response takes longer to respond to the first exposure to an antigen, the memory cell recall response to a previously encountered antigen is swift and potent.

The importance of the memory recall response is demonstrated by the SARS-CoV-2 vaccines developed for protection against COVID-19 infection. A two dose series of Moderna's mRNA-1273 vaccine administered 28 days apart had an efficacy of 94% in protecting against symptomatic infection compared with placebo.¹ This vaccine is an mRNA vaccine encoding the S-protein of the SARS-COV-2 Wuhan strain. The mRNA is translated to S-protein, which is expressed by various cells in the vaccine recipient. Upon initial exposure to the S-protein, naïve B and T-cells become stimulated by the S-protein antigen and move to local lymph nodes where they begin proliferating and differentiating to memory cells and plasma cells (long lived antibody producing cells). Plasma cells produce protective anti-S-protein antibodies and memory T-cells with anti-S-protein TCRs are primed to respond quickly to S-protein re-exposure.

Thus, in vitro assays that quantify antigen specific adaptive immune responses is highly desired. For example, SARS-COV-2 vaccine induced immune responses are likely multifaceted involving both humoral (B-cell) and cellular (T-cell) immunity. Numerous studies have evaluated both the humoral and cellular immune responses elicited by the SARS-COV-2 vaccine. Quantification of these immune responses, also called immunologic correlates of protection, can indicate an individual's protection from SARS-COV-2 infection and can allow for efficient vaccine development, evaluation of immunity towards novel variants and optimized vaccine dosing. The SARS-COV-2 vaccines were developed and evaluated in large randomized clinical trials with infection as the primary endpoint. This approach was necessary but time-consuming and costly, requiring more than 30,000 participants for each trial. Future vaccine efficacy trials face additional challenges. These trials will require non-inferiority design with existing vaccines as the standard-of-care comparator instead of placebo, further increasing the required study size. As the Omicron variant has shown, vaccine efficacy will need to be re-evaluated against new variants quickly. Future trials will need to account for immunity due to infection as well as vaccination. Finally, vaccine efficacy trials are powered to determine protection from infection, but the more important endpoint, protection from severe disease, is difficult to assess with these trials.

The establishment of an immunologic correlate of protection for SARS-COV-2 infection and severe disease would alleviate many of these challenges. A correlate of protection as a vaccine efficacy endpoint would allow for smaller clinical trials involving a few hundred participants. To determine efficacy against a novel variant, blood samples from previously vaccinated individuals can be evaluated for the immunologic correlate, instead of enrolling participants in a new clinical trial. A correlate of protection may also improve our vaccination strategy, particularly for immunocompromised individuals, who have variable responses to the vaccine, and remain at high risk for poor outcomes from COVID-19.

Recent studies²⁻⁴ have focused on humoral responses as a correlate of protection since they are significantly easier to measure compared with cellular responses. However, there is strong evidence for the importance of cellular responses in COVID-19, particularly with respect to the most important outcome, protection from severe disease⁵⁻⁷. Cellular responses to SARS-COV-2 are often measured using flow cytometry with intracellular staining or Elispot. These assays are labor intensive and require expertise to run and standardize, limiting their widespread use as a correlate of protection. The development of a reliable, inexpensive and easy to perform cellular response assay could significantly improve our ability to determine an individual's level of protection from SARS-COV-2 infection and severe disease. Such a cellular response assay could inform us of an individual's immune response to countless infectious microorganisms including but not limited to influenza, respiratory syncytial virus (RSV), parainfluenza, seasonal colds, human immunodeficiency virus (HIV), cytomegalovirus (CMV), Epstein-Barr virus (EBV), as well as numerous bacteria and fungi.

In addition to responding to infectious microorganisms, adaptive immune responses are elicited continuously in health and disease. These immune responses are critical for controlling cancer development by eliminating malignant and pre-malignant cells. Undesired immune responses can also occur in the context of various autoimmune diseases, and following transplantation of foreign tissue from organ donors. For example, a cellular response assay could serve as a prognostic marker after cancer diagnosis, determine immune responses to treatment or in the case of solid organ transplantation, determine immune responses to the transplanted organ. One of the biggest challenges in solid organ transplantation is controlling the immune response. Whereas under-immunosuppression leads to acute rejection, over-immunosuppression leads to infection, malignancy and toxicity. Both acute rejection and respiratory infections are the major risk factors for chronic rejection or chronic lung allograft dysfunction (CLAD) development. Despite the advances made in transplant medicine, artisans lack effective methods for assessing the adequacy of immunosuppression in transplant recipients. This is particularly problematic given the wide variability in the pharmacokinetics and pharmacodynamics of immunosuppression medications which is known to exist between patients. For lung transplant recipients, the standard for assessing overall immune function is histopathologic evaluation of the allograft with bronchoscopy and transbronchial biopsy, a procedure that carries a small but real risk of morbidity. Immunosuppressant drug levels (e.g., tacrolimus) are usually monitored to minimize drug related toxicities, but have poor correlation with clinical status (i.e., acute rejection or respiratory infection).

A reliable non-invasive method of assessing the adequacy of immunosuppression after lung transplantation would significantly improve outcomes for these patients. Because allo-reactive T-cells are thought to be central mediators of acute and chronic rejection, there has been increasing interest in the development of assays monitoring T-cell allo-reactivity. As noted above, a comprehensive biological assay for lymphocyte activation, one that is indicative of a broader assemblage of lymphocyte activation mechanisms and events, is needed. The present invention fulfills these needs and satisfies additional objects and advantages, as will become apparent from the following description.

### SUMMARY OF THE INVENTION

As discussed below, we have designed novel assays of lymphocyte stimulation that improve upon conventional assays of immune responses in a number of ways. The assays of the invention have a number of embodiments including methods of observing an immune response to any one of a wide variety of antigens. Typically, such methods include the steps of obtaining lymphocytes from the whole blood of a subject to be tested; incubating the lymphocytes with an antigen; observing concentrations/levels of one or more cytokines (e.g., CXCL9, CXCL10, interferon-γ, IL-2, tumor necrosis factor α, IL-18, IL-4, IL-5, IL-9, IL-13, IL-1, IL-6, IL-17, IL12, CCL20, IL10, IL-35, tumor growth factorβ, among other cytokines) released from the lymphocytes in response to incubation with the antigen; and then comparing concentrations/levels of the one or more cytokines released from the lymphocytes in response to incubation with the antigen to concentrations/levels of the same one or more cytokines released from control lymphocytes (where the control lymphocytes comprise lymphocytes from the whole blood of the subject that are not incubated with antigen). In such methods, an immune response is observed when concentrations/levels of the one or more cytokines released from the lymphocytes in response to incubation with the antigen are significantly higher (e.g., at least two, five or ten-fold greater) than concentrations/levels of the one or more cytokines released from the control lymphocytes. In certain embodiments of the invention, concentrations/levels of at least two or three or more different cytokines are observed.

Embodiments of the invention can be used to observe an immune response to a wide variety of antigens known in the art. For example, embodiments of the invention can be used to observe the immune response to a polypeptide such as a protein or a pool of overlapping peptides (e.g., those spanning a portion of protein used in a vaccine composition) or the like. In other embodiments of the invention, the antigen comprises a cell such as an allogeneic cell from an organ transplantation donor. In other embodiments of the invention, the antigen comprises major histocompatibility (MHC) molecules from potential and actual organ transplantation donors. In other embodiments of the invention, the antigen comprises an infectious agent. In some embodiments of the invention, the methods of observing concentrations/levels of one or more cytokines released from the lymphocytes include the steps of binding a cytokine with a capture antibody in an assay device comprising microfluidic channels, wherein the capture antibody is coupled to one or more regions within the microfluidic channels (such cytokines can be measured on a number of platforms including ELLA, ELISA, luminex, OLINK, SOMA etc.).

Embodiments of the invention can be used to observe an immune response in a variety of different contexts. For example, in certain embodiments of the invention, the subject is selected to be a patient immunized with a vaccine (e.g., a COVID-19 vaccine). In some embodiments of the invention, the subject is selected to be a patient who has undergone a cell or tissue transplantation procedure. In some embodiments of the invention, the subject is selected to be a patient diagnosed as having an infectious disease. In some embodiments of the invention, the subject is selected to be a patient diagnosed as having an immune disorder. In some embodiments of the invention, the subject is selected to be a patient administered an immunomodulatory agent. In some embodiments of the invention, the subject is selected to be a patient diagnosed with a malignancy.

Embodiments of the invention also include systems for observing immune response in an individual, the systems that may comprise: an antibody that binds to a CXCL9 polypeptide and an agent selected for its ability to image the antibody bound to the CXCL9 polypeptide; an antibody that binds to a CXCL10 polypeptide and an agent selected for its ability to image the antibody bound to the CXCL10 polypeptide; and an antibody that binds to a IFN-γ polypeptide and an agent selected for its ability to image the antibody bound to the IFN-γ polypeptide. Optionally, the system comprises at least one of: a detection antibody that binds to a CXCL9 polypeptide, wherein the detection antibody is coupled to a detectable label; and a capture antibody that binds to a CXCL9 polypeptide wherein the capture antibody is coupled to a matrix; a detection antibody that binds to a CXCL10 polypeptide, wherein the detection antibody is coupled to a detectable label; and a capture antibody that binds to a CXCL10polypeptide wherein the capture antibody is coupled to a matrix; and a detection antibody that binds to a IFN-γ polypeptide, wherein the detection antibody is coupled to a detectable label; and a capture antibody that binds to a IFN-γ polypeptide wherein the capture antibody is coupled to a matrix. In certain embodiments of the invention, the system comprises microfluidic channels and the detection antibody is coupled to one or more regions in the microfluidic channels. In certain of these embodiments, fluid test samples and reagents are directed by pneumatic pistons and valves through the microfluidic channels wherein the microfluidic channels are configured to perform at least three sandwich ELISAs.

Embodiments of the invention include methods of observing an immune response, the methods comprising obtaining a fluid sample selected to include CXCL9, CXCL10, IFN-γ, IL-2, tumor necrosis factor α, IL-18, IL-4, IL-5, IL-9, IL-13, IL-1, IL-6, IL-17, IL12, CCL20, IL10, IL-35, tumor growth factor β, among other cytokines, wherein the cytokines were generated by immune cells as part of an immune response (immune response cytokines); disposing the fluid sample in a Cytokine Response Assay (CRA) system disclosed herein so that concentrations of the at least one of the immune response cytokines are observed; and then correlating concentrations of at least one of these cytokines that are determined with an immune response, such that an immune response is observed. Typically in such methods, the immune response comprises an immune response to a vaccine composition. In other embodiments, the immune response comprises an immune response to transplanted tissues. In other embodiments, the immune response comprises an immune response to an infectious agent. In other embodiments, the immune response comprises an immune response to a malignancy. In certain embodiments of the invention, the immune cells are obtained from a subject administered a therapeutic agent or an immunomodulatory agent.

A number of illustrative embodiments of the invention are disclosed below including methods that can be used identify key cytokines involved in T cell responses following vaccination with viral antigens. In addition, as disclosed below, in studies of a murine tracheal transplant model as well in human lung transplant recipients, embodiments of the invention demonstrated a strong amplification of signaling downstream of IFN-γ after lymphocyte re-stimulation with donor cells. In these studies, we evaluated over 50 cytokines/chemokines involved in alternate pathways (e.g., Th2, Th17) as part of an analysis of optimal biomarkers of immune activation/stimulation. From these studies we discovered that observing the biomarker performance of three CXCR3 receptor ligands (CXCR3 ligands): CXCL9, CXCL10 and CXCL11 unexpectedly provided superior results over all other biomarkers studied herein for providing information on lymphocyte stimulation associated with the immune response.

Building upon our initial discoveries, we found that while IFN-γ concentrations were very low, the concentrations of the downstream CXCR3 ligands in culture supernatants after lymphocyte stimulation are markedly elevated, indicating an augmentation of the signal downstream. Harnessing this information, we developed assays for quantifying CXCR3 ligand concentrations as a measure of lymphocyte stimulation (e.g., concentrations in the supernatant, concentrations in blood plasma, CXCR3 ligand ELISPOT, intracellular staining for CXCR3 ligands and the like). The assays disclosed herein can be used in methods for evaluating immune response in a variety of contexts, including immune responses to transplanted donor tissue, immune responses to infectious agents such as SARS-COV-2, immune responses to vaccinations or other proteins of interest, immune responses to tumor cells and immune responses to non-specific stimulators of the immune system (e.g., immune response to the non-specific lymphocyte stimulators including, but not limited to phytohemagglutinin [PHA], phorbaol 12-myristate 13-acetate [PMA], lysophosphatidylcholine [LPS]).

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1 provides data from studies of anti-RBD IgG levels following SARS-CoV-2 mRNA vaccination in healthy controls and lung transplant recipients. “Responder” defined as anti-RBD IgG>100.

FIG. 2. provides data from studies of longitudinal CD8+ T cell response to SARS-COV-2 S-protein peptides measured by IFNγ ELISpot after first and second (arrow) BNT162b2 doses. Open circle, CD8+ T cells (left axis); “X”, Anti-RBD IgG (right axis).

FIGS. 3A and 3B provides data from studies of cross-sectional CD8+ T cell responses to SARS-COV-2 S-protein peptides measured by IFNγ ELISpot among 25 participants after SARS-COV-2 mRNA vaccination. Open circle, BNT162b2; triangle, mRNA-1273. FIG. 3A shows data following vaccine dose #1 and FIG. 3B shows data following vaccine dose #2.

FIG. 4 provides data from studies of cytokine profiling of CD4+ and CD8+ T cells after PMA/ionomycin stimulation in lung transplant recipients (Tx) and healthy participants (control).

FIG. 5 shows a schematic of a Th1 immune response to SARS-COV-2 peptide with CXCL9 signal amplification in a positive feedback loop. DC=dendritic cell, Tm=memory T cell.

FIG. 6 shows an Ella microfluidic-based automated ELISA platform including the device (left panel), the front and back of a plate for use in the device (middle panel) and a schematic of antibody binding within the device (right panel).

FIG. 7: provides data from studies illustrating the high precision of the Ella microfluidic-based automated ELISA platform at low IL-10 (sample 1) and IL-6 (sample 2) cytokine concentrations.

FIG. 8: provides data from studies illustrating the improved sensitivity of Ella microfluidic-based automated ELISA platform as compared with conventional ELISA assays.

FIGS. 9A and 9B provide data from studies of CXCL9 concentrations in unstimulated and S-protein stimulated whole-blood from healthy volunteers before and after the third vaccine dose. FIG. 9A shows data from studies pre-vaccine #3 (Median CXCL9 concentration (pg/ml): Unstimulated: 308, and Stimulated: 1664) and FIG. 9B shows data from studies post-vaccine #3 (Median CXCL9 concentration (pg/ml): Unstimulated: 296, and Stimulated: 7561, p<0.01).

FIG. 10 provides data from studies of background subtracted (S−N) CXCL9 responses from healthy volunteers before and after the third vaccine dose. Median CXCL9 concentration (pg/ml): Pre-vaccine: 1234 and Post-vaccine: 7320, p<0.01.

FIG. 11 provides data from studies of CXCL9 responses (S−N) after S-protein and N-protein simulation in a healthy participant after the third vaccine dose.

FIG. 12. provides data from studies of background subtracted (S−N) CXCL9 responses from healthy controls and lung transplant recipients before and after the third vaccine dose. Post-Vaccine Median CXCL9 concentration (pg/ml): in Healthy: 7320, and in Lung transplants: 109, p<0.01.

FIGS. 13A and 13B provide data from studies of background subtracted CXCL9 responses to S-protein in lung transplant recipients who received a 50 μg (n=8) or 100 μg (n=11) mRNA-1273 booster (fourth or fifth) dose. FIG. 13A shows data from studies with a first concentration (50 μg) of booster vaccine (Median CXCL9 concentration (pg/ml): Day 0: 61, and Day 30:106, p=0.18) and FIG. 13B shows data from studies with a second concentration (100 μg) of booster vaccine (Median CXCL9 concentration (pg/ml): Day 0: 14, and Day 30: 102, p=0.81).

FIG. 14 provides data from studies of background subtracted (S−N) CXCL9 responses to Influenza H1N1 HA in lung transplant recipients enrolled in the high-dose mRNA-1273 booster trial. Median CXCL9 concentration (pg/ml): Day 0: 1817 and Day 30: 1669, p=0.13).

FIG. 15 provides data from studies of CXCL9 response (S−N, left axis) vs. Anti-RBD IgG response (right axis) in a previously healthy participant after SARS-CoV-2 infection.

FIG. 16 provides data from studies of CXCL9 response (S−N, left axis) vs. Anti-RBD IgG response (right axis) in a previously healthy participant after SARS-CoV-2 infection.

FIG. 17 provides data from studies of allo-specific CXCR3 ligand responses in murine heterotropic tracheal transplant model.

FIG. 18 provides data from studies of allo-specific CXCR3 ligand responses in human lung transplant recipients.

### DETAILED DESCRIPTION OF THE INVENTION

In the description of embodiments, reference may be made to the accompanying figures which form a part hereof, and in which is shown by way of illustration a specific embodiment in which the invention may be practiced. It is to be understood that other embodiments may be utilized, and structural changes may be made without departing from the scope of the present invention.

As disclosed below, the invention has a number of embodiments. Embodiments of the invention include assays for quantifying various cytokine (e.g. CXCR3 ligand) concentrations as a measure of antigen-specific cellular immune response (T cell recall response). The assays disclosed herein can be used in methods for evaluating immune response in a variety contexts, including immune responses to transplanted donor tissue, immune responses to infectious agents such as COVID-19, immune responses to vaccination and other proteins of interest, immune responses to tumor/cancer cells and immune responses to non-specific stimulators of the immune system (e.g., immune response to the non-specific T-cell mitogen, phytohemagglutinin). Embodiments of the invention include assays that quantify cytokine and cytokine panels including, but not limited to CXCL9, CXCL10, interferon-γ, IL-2, tumor necrosis factor α, IL-18, IL-4, IL-5, IL-9, IL-13, IL-1, IL-6, IL-17, IL12, CCL20, IL10, IL-35, tumor growth factor β, as a measure of antigen-specific cellular immune response (T cell recall response).

Embodiments of the invention also include methods of observing an immune response to an antigen. Typically the methods comprise obtaining immune cells (including, but not limited to lymphocytes, monocytes and macrophages, basophils, neutrophils, and eosinophils, as well as antigen presenting cells such as dendritic cells) from a subject (e.g. from the whole blood of the paitent); and then incubating the immune cells that are obtained with the antigen. The methods include observing concentrations of one or more cytokines released from the immune cells in response to incubation with the antigen; and then comparing concentrations of the one or more cytokines released from the immune cells in response to incubation with the antigen to control immune cells, wherein the control immune cells comprise the same population of immune cells from the whole blood of the subject, but which are not incubated with antigen. The methods further include observing the presence or absence of an immune response, wherein an immune response is observed when concentrations of the one or more cytokines released from the immune cells in response to incubation with the antigen are at least two-fold greater than concentrations of the one or more cytokines released from the control immune cells.

Embodiments of the invention can be used to observe an immune response to a wide variety of antigens known in the art. For example, embodiments of the invention can be used to observe the immune response to a polypeptide such as a protein or a pool of overlapping peptides (e.g., those spanning a portion of protein used in a vaccine composition) or the like. In other embodiments of the invention, the antigen comprises a cell such as a malignant cell from a patient with cancer. In other embodiments of the invention, the antigen comprises a cell such as an allogeneic cell from a transplantation donor. In other embodiments of the invention, the antigen comprises an infectious agent or a portion thereof. Typically in such embodiments, lymphocytes obtained from the subject are incubated with the antigen for at least 5, 10 or 20 hours.

Embodiments of the invention can be used to observe an immune response in a variety of different contexts. For example, in certain embodiments of the invention, the subject is selected to be a patient immunized with a vaccine. In some embodiments of the invention, the subject is selected to be a patient who has undergone a cell or tissue transplantation procedure. In some embodiments of the invention, the subject is selected to be a patient diagnosed as having an infectious disease. In some embodiments of the invention, the subject is selected to be a patient diagnosed as having an immune disorder. In some embodiments of the invention, the subject is selected to be a patient administered an immunomodulatory agent. In some embodiments of the invention, the subject is selected to be a patient diagnosed with a malignancy.

Certain embodiments of the invention are directed to assays designed to observe immune responses to transplanted donor tissue. Because allo-reactive T-cells are thought to be central mediators of acute and chronic rejection, there has been increasing interest in the development of assays monitoring T-cell allo-reactivity. The Immuknow assay measures ATP production by CD4 T-cells after stimulation with the non-specific T-cell mitogen, phytohemagglutinin. It is one of the only FDA approved assays for assessing immune function after solid organ transplantation. However, our group as well as others have demonstrated that this assay has poor sensitivity, specificity and overall clinical utility. The ELISPOT assay stimulates recipient lymphocytes with inactivated donor lymphocytes on an interferon-γ (IFN-γ) capture plate. The IFN-γ“spots” represent stimulated allo-sensitized T-cells and are quantified. Several studies in kidney transplantation have reported an association between pre-and post-transplant

ELISPOT measurements and acute rejection/graft function. Further studies assessing its utility are needed, but this assay is limited clinically due to its poor sensitivity, labor-intensiveness, variability and difficulty with standardization.

One of the biggest challenges in solid organ transplantation is controlling the immune response. Whereas under-immunosuppression leads to acute rejection, over-immunosuppression leads to infection, malignancy and toxicity. Both acute rejection and respiratory infections are the major risk factors for chronic rejection or chronic lung allograft dysfunction (CLAD) development. Despite the advances made in transplant medicine, artisans lack effective methods for assessing the adequacy of immunosuppression in transplant recipients. This is particularly problematic given the wide variability in the pharmacokinetics and pharmacodynamics of immunosuppression medications which is known to exist between patients. For lung transplant recipients, the standard for assessing overall immune function is histopathologic evaluation of the allograft with bronchoscopy and transbronchial biopsy, a procedure that carries a small but real risk of morbidity. Immunosuppressant drug levels (e.g. tacrolimus) are usually monitored to minimize drug related toxicities, but have poor correlation with clinical status (i.e., acute rejection or respiratory infection). In this context, embodiments of the invention disclosed herein provide reliable non-invasive methods of assessing the adequacy of immunosuppression after lung transplantation would significantly improve outcomes for these patients.

Some embodiments of the invention are directed to assays designed to observe immune responses to vaccinations. Certain embodiments of the invention are directed to assays designed to observe immune responses to infectious agents such as viruses, bacteria, fungi and parasites. In the working embodiments discussed below, the infectious agent is COVID-19.

A lymphocyte stimulation assay that can quantify an individual's cellular immune response can allow for efficient vaccine development, evaluation of immunity towards novel variants and optimized vaccine dosing. In one embodiment of our invention, we developed a novel whole blood SARS-COV-2 T-cell stimulation assay that quantifies antigen specific T-cell responses in vitro, and is easy to run with minimal labor. The assay relies on the addition of SARS-COV-2 peptides into whole blood. During an incubation/stimulation period, SARS-COV-2 peptides are processed and presented by antigen presenting cells to memory T-cells that respond with a rapid and robust immune response that can be measured by the amount of cytokines released.

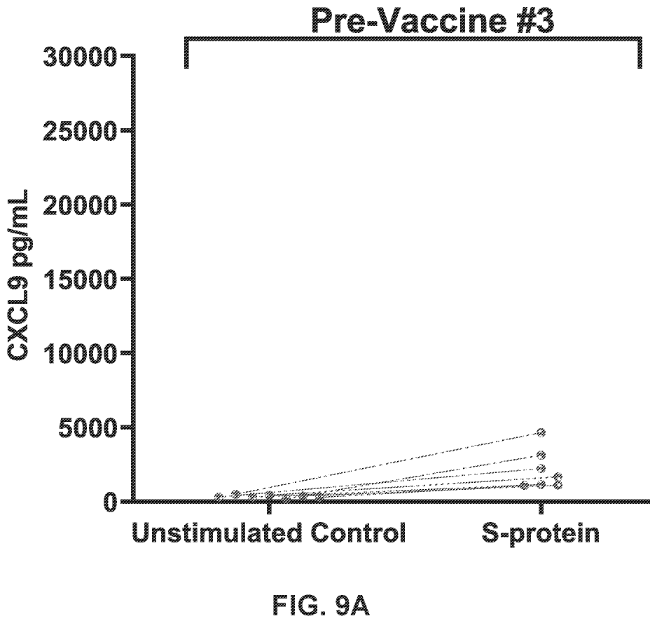

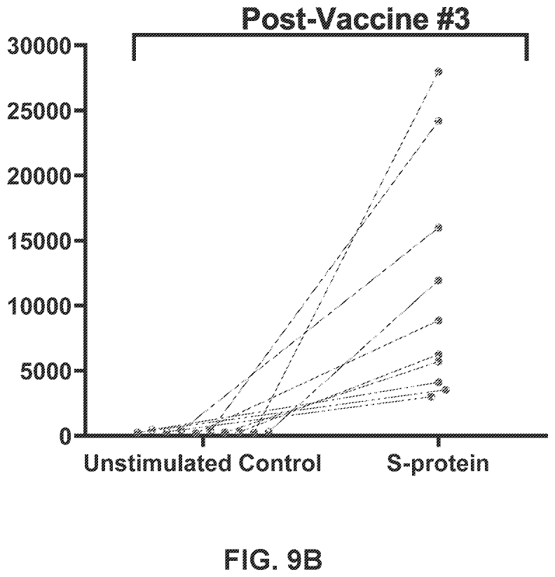

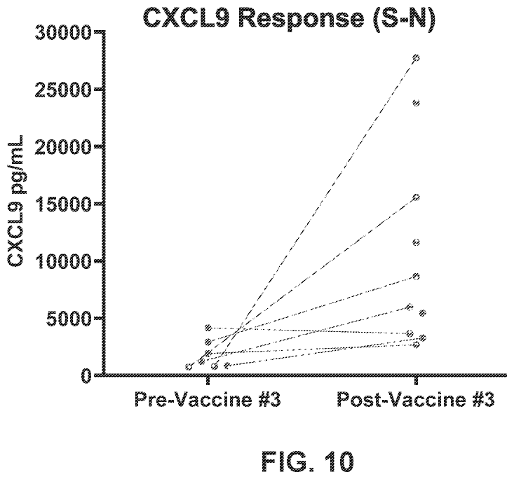

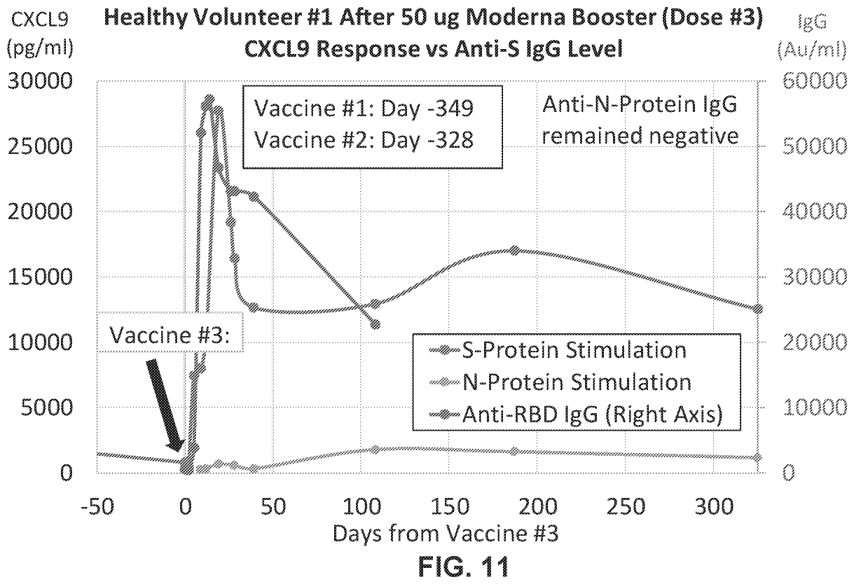

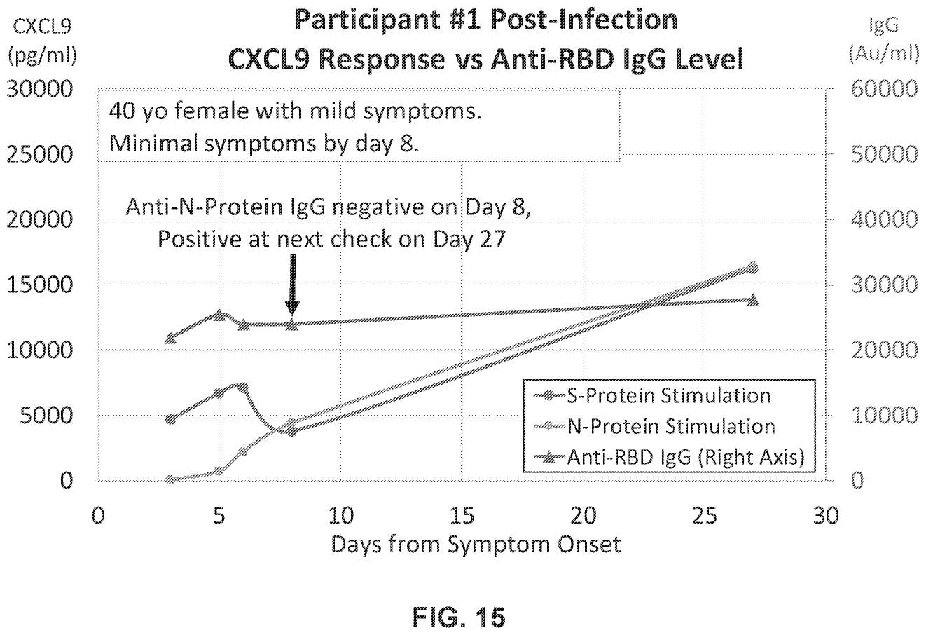

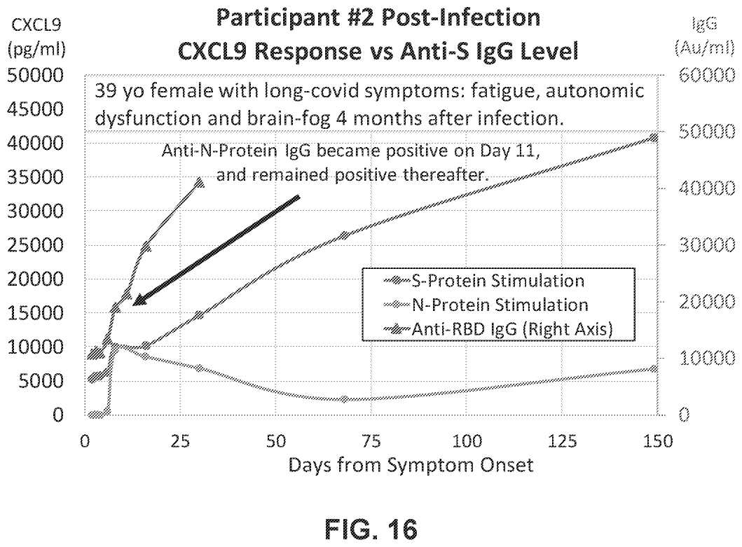

Among healthy volunteers after SARS-COV-2 mRNA vaccination, we evaluated T-cell responses after a 20-hour stimulation with SARS-COV-2 antigens, as well as negative controls incubated for 20 hours without peptide stimulation. We found significant signal amplification in a chemokine downstream of IFN-γ, CXCL9, in response to T-cell stimulation with SARS-COV-2 proteins. CXCL9 is induced by IFN-γ and acts as a potent chemoattractant for mononuclear cells (e.g., CD4 and CD8 T-cells). For the negative control samples without peptide stimulation, the median CXCL9 concentration was 308 pg/mL compared with a median IFNγ concentration of 0.47 pg/mL. Among volunteers who received only two vaccine doses, the median CXCL9 concentration was 308 pg/mL for the negative controls, compared with 1664 pg/mL with S-protein (2.5 μg/mL) stimulation (FIG. 9A). Among volunteers who received three vaccine doses, the median CXCL9 concentration was 296 pg/mL for the negative controls, compared with 7561 pg/mL with S-protein stimulation (FIG. 9B). CXCL9 signal minus noise (S−N) was calculated as follows: CXCL9 concentration from S-protein (2.5 μg/mL) stimulated samples-CXCL9 concentration from negative controls (FIG. 10). There was variability noted in these CXCL9 S−N responses between volunteers with some showing a robust increase in CXCL9 levels after vaccine #3, while others showing no significant CXCL9 increase. FIG. 11 shows the robust CXCL9 increase after SARS-COV-2 stimulation in a healthy volunteer before and after the third vaccine dose. CXCL9 concentrations began to increase on Day 6 after vaccination and appeared to level off/decrease 20 days after the dose. Of note, there was no significant response to SARS-COV-2 N-protein stimulation for this volunteer who was not previously infected with SARS-COV-2. N-protein is a SARS-COV-2 protein that is found in the virus, but not the mRNA vaccine. FIGS. 15 and 16 shows post-stimulation CXCL9 concentrations in two volunteers (vaccinated×3) who became infected with SARS-COV-2. CXCL9 levels in response to S-protein stimulation began to increase 6 days after SARS-COV-2 infection and appeared to be increasing at day 150 (FIG. 16). CXCL9 responses to N-protein were initially low, but began to increase on Day 6.

By stimulating T-cells in whole blood with SARS-COV-2 peptides and measuring CXCL9 release, we are able to quantify a robust T-cell response signal to any SARS-COV-2 component of interest. The disclosure provided herein provides evidence that this novel assay will provide a simple, rapidly deployable and reliable mechanistic correlate of protection for SARS-COV-2. Embodiments of the invention can be used to determine the correlates of protection from infection and severe disease using a whole blood T-cell SARS-COV-2 stimulation assay.

Embodiments of the invention can be used to determine the correlates of protection from infection and severe disease for other respiratory viruses including influenza, respiratory syncytial virus, parainfluenza, seasonal cold viruses, as well as non-respiratory microorganisms including HIV, CMV, EBV, as well as numerous bacteria and fungi.

### Illustrative Protocol for SARS-COV-2 and Influenza T-cell Stimulation Assay

1. Collect blood in 6 mL Lithium Heparin tubes.
2. Gently invert the tube 8 times to mix lithium heparin with blood.
3. Within 6 hours of blood collection, aliquot blood into 7-0.8 mL aliquots in sterile culture tubes.
4. Add the following to the 7 tubes of blood as follows:
   a. Negative Control—no stimulant added.
   b. PHA at amounts including 2.5 ug, 5.0 ug and 7.5 ugs.
   c. S-protein (e.g. Miltenyi SARS-COV-2 S-protein Complete Peptivators, and other SARS-COV-2 proteins, peptides and peptide pools) at amounts including 2.5 ug, 5 ug, 7.5 ugs.
   d. Influenza protein H1N1 HA antigen (e.g. Miltenyi's Influenza H1N1 Hemaglutinin A Peptivator, and other influenza proteins, peptides and peptide pools) at amounts including 2.5 ug, 5 ug, 7.5 ugs.
   e. N-protein (e.g. Miltenyi SARS-COV-2 N-protein Peptivator, and other SARS-COV-2 proteins, peptides and peptide pools) at amounts including 2.5 ug, 5 ug, 7.5 ugs.
5. Gently mix tubes by gentle pipetting—3 times using 1 mL pipette.
6. Incubate 16-24 hours at 37 degrees C.
7. After 16-24 hour incubation, spin culture tubes at 300 g×10 mins.
8. Aliquot supernatant (plasma) into 4 epindorf tubes—200 μL each.
9. Freeze at −80 C for future cytokine measurement.

### Illustrative Protocol for CXCL9 Measurement on Ella ProteinSimple:

1. Thaw plasma at room temp.
2. Within 6 hours of thawing, mix plasma 50-50 with CXCL9 Cartridge standard diluent (included with cartridge).
3. Pipette 50 μL of plasma-diluent mix into appropriate well on 72 well cartridge.
4. Add 1mL of buffer (included) to all buffer wells.
5. Remove plastic adhesive from bottom of cartridge.
6. Place cartridge into cartridge slot.
7. Press start on SimplePlex Runner software.
8. Results will be ready in 80 mins.

### Illustrative SARS-COV-2 T-cell Assay Data Interpretation:

1. “Stimulation” will be measured as MIG concentration in SARS-COV-2 stimulated sample—MIG concentration in negative control.
2. We will determine what the clinically relevant “Stimulation” threshold is for the “Correlate of Protection” against infection and severe disease. This can be determined using clinical studies of individuals before SARS-COV-2 infection and evaluation of their outcomes after infection.
3. Samples with the following can be considered indeterminate results.
   a. Negative control MIG level >=800.

As noted above, the disclosure provided herein includes assays of immune responses that provide elegant, unique and very powerful designs. These assays will allow accurate and reliable measurements of cellular (T cell) immune responses against viral pathogens including SARS-COV-2, influenza, HIV, CMV etc.

Our assay provides several advantages to existing assays that quantify cellular immune responses. Cellular responses to SARS-COV-2 are often measured using enzyme-linked immunosorbent spot (ELISpot) or flow cytometry with intracellular cytokine staining (ICS). These assays measure the frequency of responding T cells, but are limited by several shortcomings including: 1) the need for many peripheral blood mononuclear cells (PBMCs); 2) the inability to determine the magnitude of cytokine release from cells; 3) the inability to phenotype cytokine secreting cells; 4) a labor-intensive procedure; and 5) high costs. ICS offers several advantages over ELISpot, such as the ability to obtain phenotypic details of cytokine-producing cells including cell type and activation status, and the ability to track multiple cytokines (i.e. polyfunctional cells). However, ICS remains limited by: 1) poor sensitivity to detect low-frequency responses, especially with limited PBMC counts (e.g. from individuals on immunosuppressive medications); 2) the inability to determine the magnitude of cytokine release; 3) the number of cytokines that can be tracked concurrently; 4) a labor-and expertise-intensive procedure; and 5) high costs. ICS requires that the cytokine detection be pre-determined, with standard panels often using interferon-gamma (IFNγ), interleukin (IL)-2, and tumor necrosis factor-alpha (TNFα). T cell responses to vaccination however, are heterologous and complex, and measurements based on only a few cytokines may significantly underestimate the response.

The assay disclosed herein has several advantages compared with both ELISpot and ICS including: 1) inexpensive; 2) improved sensitivity; 3) physiologic stimulation involving all whole blood components; 4) the ability to evaluate the concurrent expression of hundreds of cytokines; and 5) the ability to determine the magnitude of cytokine response (in cytokine concentration [pg/ml], as opposed to the frequency of responding cells which may not capture the magnitude of the immune response).

Additionally, the assay disclosed herein is able to quantify cellular immune responses against other antigens of interest including infectious microorganisms including but not limited to influenza, respiratory syncytial virus (RSV), parainfluenza, seasonal colds, human immunodeficiency virus (HIV), cytomegalovirus (CMV), Epstein-Barr virus (EBV), as well as numerous bacteria and fungi. Furthermore, cellular immune responses against other antigens of clinical interest including but not limited to tumor/malignant cells, targets of autoimmunity (rheumatoid arthritis (RA), systemic lupus erythmatosis (SLE), etc), and organ transplant donor cells/tissues can be quantified with the disclosed assay. This can be accomplished by the addition of the antigen of interest (e.g. tumor cells/proteins, targets of autoimmune disease, and donor cells/tissues) to the assay and measuring the associated cytokine response. Additionally, the disclosed assay will allow measurement of immune response cytokine concentrations in several media including whole blood, plasma, serum, supernatant (i.e., from ELISpot and ICS procedures).

Embodiments of the invention disclosed herein fill a significant void in the currently available methods of assessing immune responses to numerous antigens which are important in human and mammalian health and disease. The invention disclosed may improve our understanding and medical management in infectious disease/vaccine medicine, oncology, autoimmune disease, lung transplantation, as well as the field of immune modulation/immune therapies.

### Example 1: Observing Cytokine Responses to Covid Vaccine Polypeptides

SARS-COV-2 mRNA vaccines induce both humoral and cellular immunity. Due to rapid evolution of the spike protein receptor binding domain (RBD), the key target of neutralizing antibodies (NAbs), vaccine-induced humoral immunity has lost activity against novel variants.⁸⁻¹¹ By contrast, T cells recognize short 8-15 amino-acid peptides encoded across the entire SARS-COV-2 genome and are not limited to targeting the rapidly evolving RBD, allowing T cells to remain effective against new variants.¹²⁻²⁰ By killing infected cells and limiting viral replication, T cells protect against severe disease,^{7,21-23} which is the primary goal of SARS-COV-2 vaccines.

T cell responses are typically measured using flow cytometry with intracellular cytokine staining (ICS) or enzyme-linked immunosorbent spot (ELISpot). These assays, however, are labor-intensive, expensive, with limited sensitivity to detect low-frequency responses, especially with limited peripheral blood mononuclear cells. Although these assays identify frequencies of cytokine-producing cells, they do not quantify cytokine production.

The Example discloses a novel T cell stimulation assay (“Cytokine Response Assay” [CRA]) to quantify SARS-COV-2 specific T cell recall responses as a sensitive, reliable, easy to measure, and rapidly scalable mechanistic correlate of protection against SARS-Cov-2. Similar to the QuantiFERON-TB Gold test, the CRA measures cytokines produced by T cells in whole blood in response to viral peptides. Using the CRA, we have identified strong signal amplification for chemokines downstream of interferon-γ (IFNγ): CXCL9 and CXCL10. These IFNγ-induced chemokines are potent chemoattractants for mononuclear cells (T cell, B cells and NK cells), and major mediators of Th1 immunity against viral infections.²⁴⁻²⁶ The CRA, by concurrently measuring many cytokines released and their magnitudes, can allow determination of the key cytokines involved in the SARS-COV-2 vaccine-induced T cell recall response.

Embodiments of the invention can be used to measure an individual's cellular immune response against SARS-COV-2 elicited due to SARS-COV-2 vaccination or infection. The CRA has several advantages over ICS and ELISpot including low cost, improved sensitivity and the ability to evaluate the concurrent expression of hundreds of cytokines. By adjusting the stimulating SARS-COV-2 peptide pools, the CRA can be rapidly updated to quantify cellular responses for any new vaccine or novel SARS-CoV-2 variant. The CRA is easily scaled-up and standardized across laboratories, allowing for decentralized sample analysis in clinical trials. Ease of use, low cost, and the production of abundant data make the CRA an efficient and effective method for studying vaccine-induced T cell recall responses in large numbers of participants.

The CRA has an important advantage over humoral assays (neutralizing and binding antibody assays) in its ability to quantify immune responses against internal viral proteins. This will allow the CRA to be used to evaluate next generation vaccines involving conserved internal components of the SARS-COV-2 virus (e.g. N-protein, ORFs, etc.), a clear advantage over humoral assays that measure binding of conformational epitopes on the SARS-COV-2 surface. Furthermore, there is increasing evidence that while NAbs mediate the rapidly waning protection against infection (due to RBD evolution of the variants), cellular responses mediate the durable protection from severe disease that is the primary goal of the SARS-COV-2 vaccines.

Thus, Embodiments of the invention will provide a sensitive and reliable assay of cellular immunity that will allow for improved efficiency in SARS-COV-2 vaccine development, evaluation of vaccine efficacy against novel variants and the identification of individuals with inadequate cellular immunity (such as the immunocompromised and organ transplant recipients) who would benefit from additional SARS-COV-2 booster vaccine doses.

Embodiments of the invention can also be used to measure cellular immune responses against numerous infectious microorganisms including but not limited to influenza, respiratory syncytial virus (RSV), parainfluenza, seasonal colds, human immunodeficiency virus (HIV), cytomegalovirus (CMV), Epstein-Barr virus (EBV), as well as bacteria and fungi. By adjusting the stimulating antigens to correspond to the microorganism of interest, embodiments of the invention can provide a sensitive and reliable measure of an individuals cellular immunity against all, but not limited to, the infectious microorganisms listed above (e.g. influenza, RSV, parainfluenza, seasonal cold, HIV, CMV, EBV, bacteria and fungi)

SARS-COV-2 mRNA vaccines induce both humoral and cellular responses. Neutralizing antibodies (NAbs) recognize and bind conformational epitopes on the SARS-COV-2 spike protein (S-protein) receptor binding domain (RBD), thereby blocking engagement with the host angiotensin-converting enzyme 2 receptor (ACE2). However, evolution of the RBD has led to escape from NAbs elicited by vaccination or prior infection, leading to breakthrough infections after vaccination, reinfections, and loss of monoclonal antibody therapeutic efficacy.^{18,27-30}

By contrast, T cells are able to recognize short 8-15 amino acid peptides encoded across the entire SARS-COV-2 genome and are not limited to targeting the rapidly evolving RBD. The ability to recognize epitopes from more conserved regions of the SARS-COV-2 genome allows T cell to remain responsive against new SARS-CoV-2 variants.^{5,12,31-34} T cells also have the important advantage of recognizing epitopes from internal proteins (e.g. nucleocapsid, ORFs etc.), compared with antibodies that only recognize conformational epitopes on the SARS-COV-2 surface. Although T cells have a limited role in preventing SARS-COV-2 infection, they can recognize and kill infected host cells, providing a powerful response that limits viral replication and minimizes disease severity. Several studies have demonstrated this role of vaccine-induced spike-specific T cells mediating protection against severe disease,^{7,21-23} the primary goal of SARS-COV-2 vaccines. This T cell mediated protection against severe disease has remained durable over time and across variants.¹²⁻²⁰

T cell responses to SARS-COV-2 are often measured using enzyme-linked immunosorbent spot (ELISpot) or flow cytometry with intracellular cytokine staining (ICS). Shortcomings of ELISpot include: 1) the need for many peripheral blood mononuclear cells (PBMCs); 2) the inability to determine the magnitude of cytokine release from cells; 3) the inability to phenotype cytokine secreting cells; 4) a labor-intensive procedure; and 5) high costs. ICS offers several advantages over ELISpot, such as the ability to obtain phenotypic details of cytokine-producing cells including cell type and activation status, and the ability to track multiple cytokines (i.e., polyfunctional cells). However, ICS remains limited by: 1) poor sensitivity to detect low-frequency responses, especially with limited PBMC counts (e.g. from individuals on immunosuppressive medications); 2) the inability to determine the magnitude of cytokine release; 3) the number of cytokines that can be tracked concurrently; 4) a labor-and expertise-intensive procedure; and 5) high costs. ICS requires that the cytokine detection be pre-determined, with standard panels often using interferon-gamma (IFNγ), interleukin (IL)-2, and tumor necrosis factor-alpha (TNFα). T cell responses to vaccination, however, are heterologous and complex, and measurements based on only a few cytokines may significantly underestimate the response. Although the ELISpot and ICS assays identify frequencies of cytokine-producing cells, they do not quantify the cytokine production, thereby missing an important component of the cellular immune response.

Embodiments of the invention include the use of a novel, whole-blood, T cell stimulation assay (cytokine response assay [CRA]) that provides several advantages over ELISpot and ICS including: 1) simplicity; 2) improved sensitivity; 3) physiologic stimulation involving all whole blood components; 4) the ability to evaluate the concurrent expression of hundreds of cytokines; and 5) the ability to determine the magnitude of cytokine response (in pg/ml).

The following sections describe our disclosure evaluating humoral and cellular immune responses after SARS-COV-2 vaccination and infection among healthy non-immunocompromised participants and lung transplant recipients. Our CRA results focus on CXCL9 responses after T cell stimulation but highlight the need to evaluate the involvement of additional Th1 cytokines as well as Th2, Th17 and Treg cytokines in the T cell recall response. Among all solid organ transplant recipients, lung transplant recipients receive one of the strongest immunosuppression regimens and have weak responses to SARS-COV-2 vaccination.³⁵⁻⁴⁰ Thus, they make an effective comparison group for evaluating SARS-COV-2 vaccine-induced immune responses.

### Compared with Healthy Individuals, Lung Transplant Recipients Have Weak and Variable Anti-RBD Antibody Responses After Vaccination

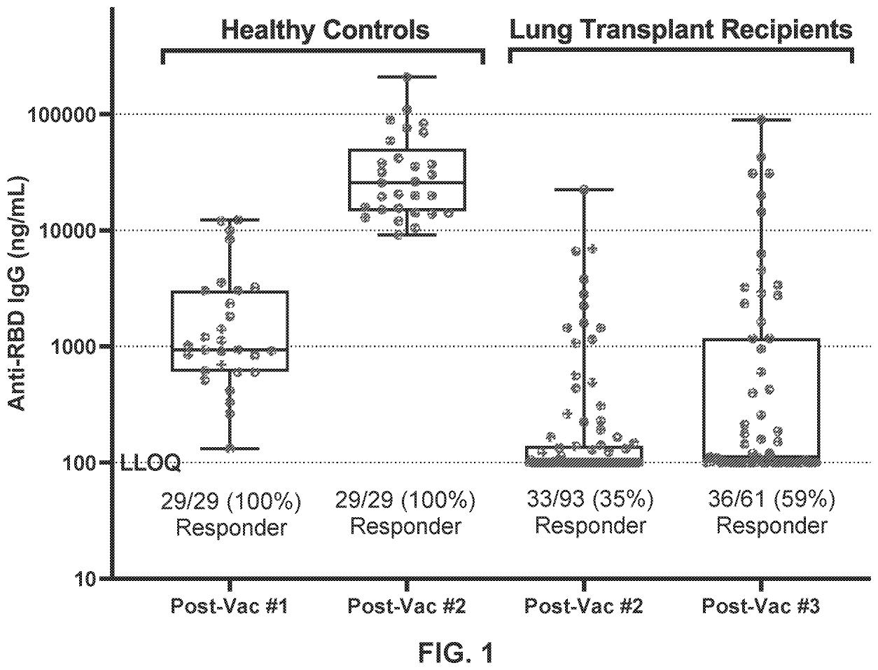

We evaluated anti-RBD IgG responses in 29 healthy SARS-COV-2 naïve participants both before and after the second vaccine dose and in 98 lung transplant recipients both before and after the third vaccine dose (FIG. 1). IgG levels were measured by enzyme-linked immunosorbent assay (ELISA) and calibrated using the monoclonal antibody CR3022, as the WHO International Serologic Standard was not available at the time. Anti-RBD IgG antibodies were detected in 100% of healthy controls after the first and second vaccine doses, with median IgG levels of 933 and 25704 ng/ml, respectively. By contrast, anti-RBD IgG antibodies were detectable in only 35% and 59% of lung transplant recipients after the second and third vaccine doses, respectively. Median IgG levels were below the lower limit of quantification (LLOQ, 100 ng/ml) after the second dose and just above the LLOQ (112 ng/ml) after the third dose. Among those with a detectable antibody response after the third vaccine dose, responses were significantly weaker than those in healthy individuals, with only 25% developing antibody levels above 1000 ng/ml.

Although transplant recipients receive standardized immunosuppression regimens based on factors that include time from transplant and age, large differences in functional immunosuppression are observed, likely due to differences in dosing, absorption, and metabolism of immune-suppressing medications, drug interactions, and the strength of each patient's underlying immune system.

### Rapid Contraction of CD8+ T Cells by IFNγ ELISpot After mRNA Vaccination in Healthy Participants

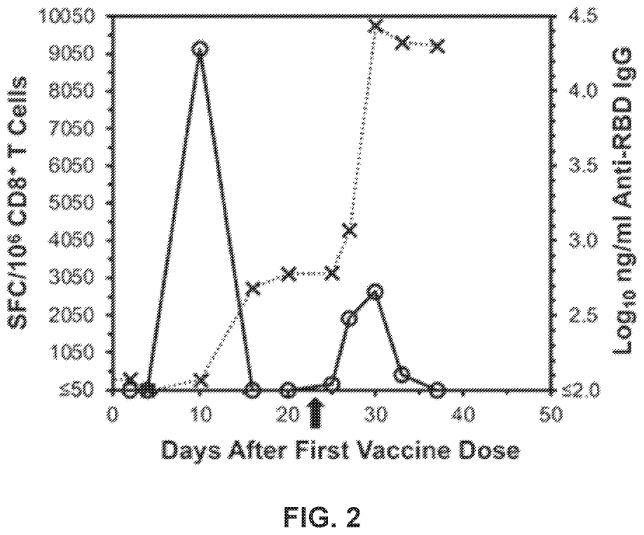

CD8⁺ T cell responses after SARS-COV-2 mRNA vaccination were evaluated by IFNγ ELISpot in 25 healthy participants from FIG. 1, using purified CD8⁺ T cells and pooled overlapping peptides spanning the S protein (BEI Resources, NR-52402) as previously described.⁴¹⁻⁴⁴ Detailed kinetics were followed longitudinally in one participant (FIG. 2) after the first and second dose (arrow) BNT162b2 vaccine. The responding CD8⁺ T cell frequency increased sharply approximately 10 days after each dose, with rapid declines shortly thereafter. These results appear particular to mRNA vaccine format, possibly due to the short-lived exposure to antigen.⁴⁵ Interestingly, the responding CD8⁺ T cell frequency after the second vaccine dose was lower than the frequency after the first dose. In contrast, anti-RBD IgG antibodies (×) followed more typical kinetics, with persistence and progressive boosting after each vaccine dose.

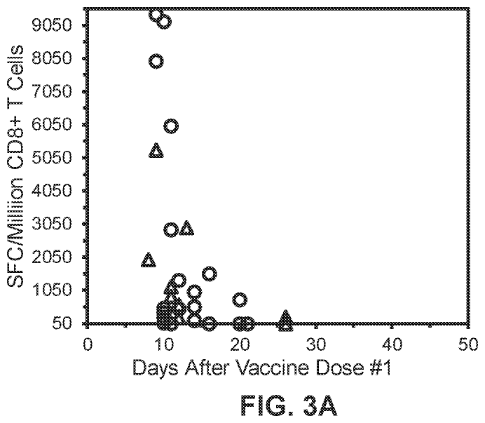

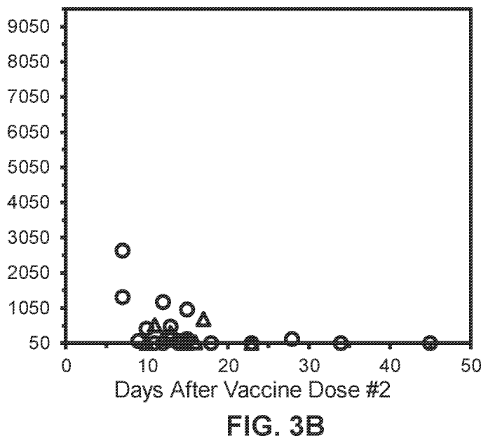

Cross-sectional evaluation of 25 healthy participants after SARS-COV-2 mRNA vaccination revealed a similar picture, with a sharp increase in the responding CD8+ T cell frequency observed 10 days after the first dose (FIG. 3, left) and a smaller peak observed 10 days after the second dose (FIG. 3, right). These findings may be explained by a transition from a naïve to a memory T cell phenotype (through clonal expansion and contraction) after the first vaccine dose. Although the frequency of responding cells is lower after the second vaccine dose, the responding cells may have a memory phenotype capable of robust responses, consistent with the clinical vaccine efficacy for protection against severe disease observed after the second vaccine dose.^{20,46-48} These findings highlight the need to consider both the frequency and the antiviral potency of responding T cells.

### Cytokine Profiling by ICS Reveals a Decreased Th1 Inflammatory Response to PMA/Ionomycin Stimulation in Lung Transplant Recipients Compared with Healthy Participants

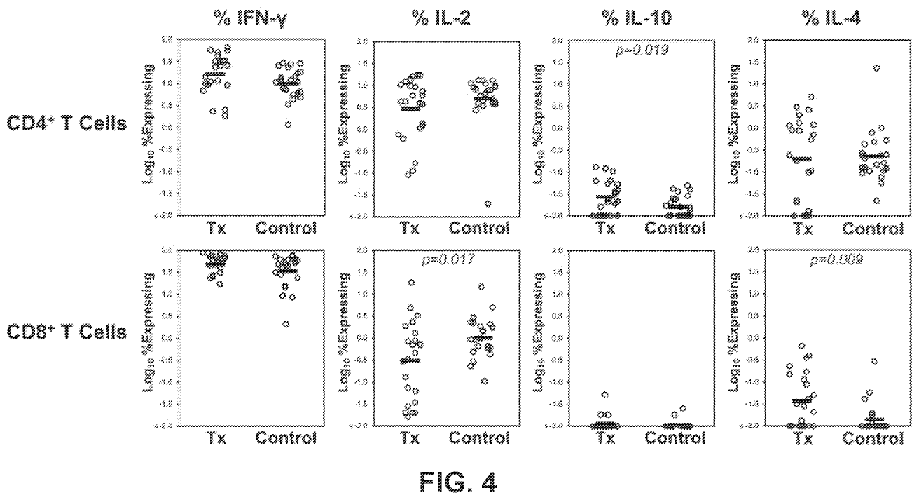

ICS was performed as previously described,⁴² to evaluate IFNγ, IL-2, IL-10, and IL-4 production by CD4⁺ and CD8⁺ T cells in response to non-specific stimulation by PMA/ionomycin, comparing 23 lung transplant recipients with 22 healthy controls. The ratios of CD4⁺ and CD8⁺ T cells were relatively similar between groups. A higher percentage of IL-10 producing cells in the CD4⁺ compartment was observed in the lung transplant group than in the healthy group (p=0.019, FIG. 4). A lower percentage of IL-2 producing cells (p=0.017) and a higher percentage of IL-4 producing cells (p=0.009) in the CD8+compartment was observed in the lung transplant group than in the healthy group. The observation of increased regulatory IL-10 produced by CD4⁺ T cells and decreased Th1-based IL-2 and increased Th2-based IL-4 produced by CD8⁺ T cells is consistent with decreased Th1 immunoreactivity and a potential shift toward Th2 and regulatory responses in lung transplant recipients compared with healthy controls. However, this ICS-based analysis of cytokine expression was limited to four cytokines reflecting Th1, Th2 and regulatory responses, providing only a partial picture of the complex and dynamic T cell response. A more comprehensive evaluation of cytokine expression profiles over time would improve our understanding of T cell recall, and the key cytokine interactions involved. In addition, as described above the magnitude of cytokine expression may be more informative than the frequency of responding T cells for understanding antiviral potency.

### A Simple Novel Whole-Blood T Cell Stimulation Assay (CRA) is Highly Quantitative and Sensitive

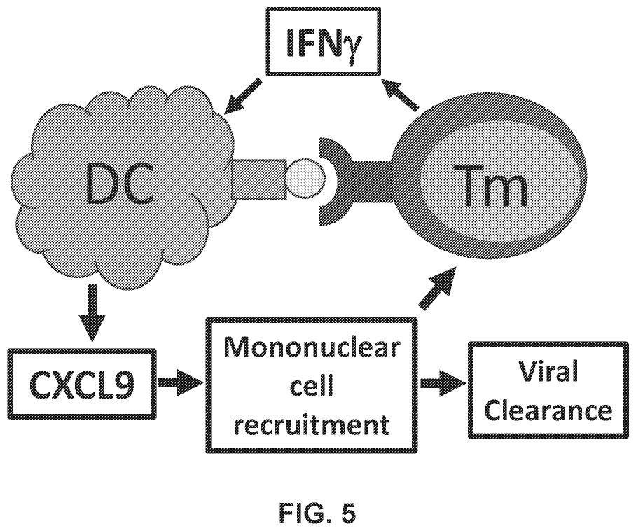

The proposed CRA relies on the stimulation of memory T cells by SARS-COV-2 peptides presented by antigen presenting cells (FIG. 5). Similar to the QuantiFERON-TB Gold test, SARS-COV-2 peptides are added to whole blood and incubated for 20 hours at 37° C. We found strong signal amplification in two chemokines downstream of IFNγ: CXCL9 (MIG) and CXCL10 (IP10). IFNγ released by T cells induces CXCL9 and CXCL10 expression by dendritic cells (DCs) in a Th1 immune response. These chemokines then act as potent chemoattractants for mononuclear cells (T cells, B cells and natural killer cells) through a common receptor, CXCR3.⁴⁹ Thus, CXCL9 and CXCL 10 release from DCs attract CXCR3 expressing T cells, resulting in further antigen presentation (Signal 1), costimulation (Signal 2), and cytokine signaling (Signal 3), the three signals required for naïve T cell activation, clonal expansion and differentiation into effector and memory T cells.⁵⁰⁻⁵² Furthermore, IFNγ release from T cells, CXCL9/CXCL10 release from DCs, and recruitment of additional T cells establishes a positive feedback loop53 that has been shown to be critically important for the clearance of several types of viral and non-viral infections.^{24-26,54} Prior studies by our group have demonstrated both the signal amplification of CXCL9, as well as the prognostic utility of CXCL9 as a plasma biomarker of Th1 immune responses during both respiratory infection55 and acute rejection⁵⁶⁻⁵⁸ in lung transplant recipients. The CRA can quantify memory T cell responses to specific SARS-COV-2 components, including internal proteins such as nucleocapsid (N-protein), a clear advantage over neutralizing antibody (NAb) assays that only quantify the binding of conformational epitopes on the SARS-COV-2 surface.

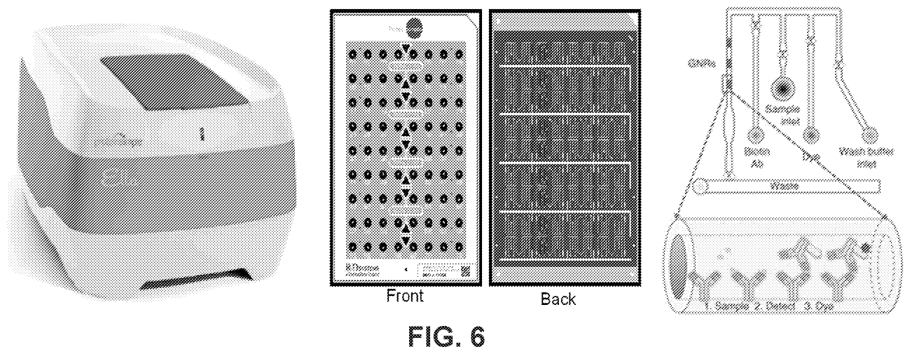

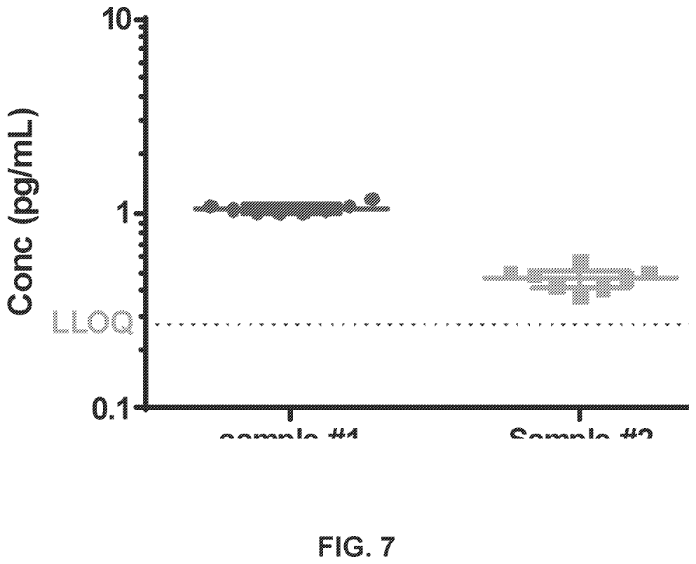

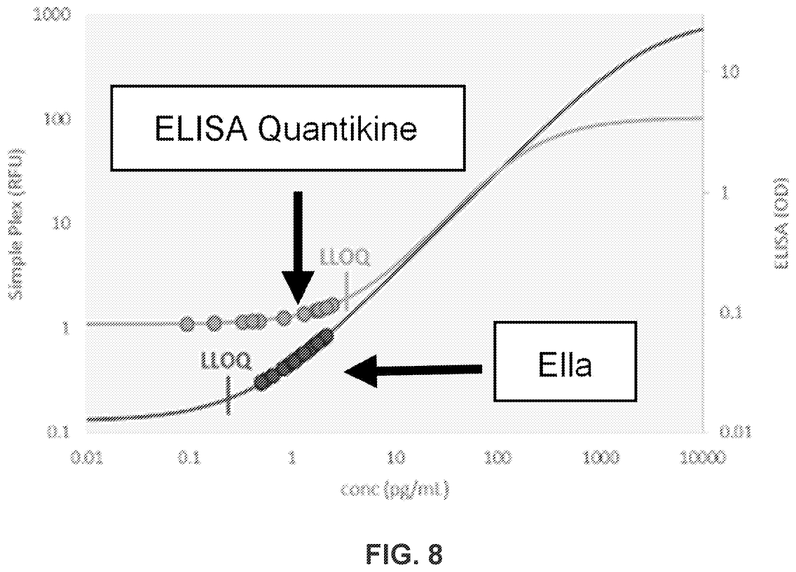

CXCL9 levels were measured using Ella, a commercially available, robust, and well-validated microfluidic ELISA platform able to run 72 samples on a single cartridge in 80 mins with no additional labor required (FIG. 6). The platform uses pneumatic pistons and values to direct the sample through microfluidic channels into three “glass nanoreactors”, where three sandwich ELISAs are captured for each sample. Thus, each analyte is detected in triplicate, improving both precision (FIG. 7) and sensitivity (FIG. 8) compared with standard ELISA platforms. Depending on the platform used for protein measurement (e.g., Olink, Simoa), the CRA can quantify the concurrent expression of hundreds of cytokines involved in the recall response.

### CXCL9 Responses Are Robust in Healthy Participants After SARS-COV-2 Vaccination

We evaluated the performance of the CRA among healthy volunteers before and after receiving the mRNA-1273 vaccine booster (Dose #3). Whole blood collected in a lithium heparin tube was divided into 0.8-ml aliquots in sterile culture tubes. Overlapping peptide pools (0.125 μg, Peptivator, Miltenyi Biotec) of the SARS-COV-2 protein of interest were added to the culture tubes and incubated for 20 hours at 37° C., followed by centrifugation. Plasma was stored at −80° C. until CXCL9 analysis. FIG. 9A shows CXCL9 levels after a 20-hour incubation in unstimulated controls and S-protein stimulated whole blood from seven healthy volunteers before the third vaccine dose (median 1-day pre-vaccine). The median CXCL9 concentration was 308 pg/ml in unstimulated samples vs. 1664 pg/ml in stimulated samples (Wilcoxon, p<0.01). After the third vaccine dose (median 45 days post-vaccine), the median CXCL9 concentration was 296 pg/ml in unstimulated samples vs. 7561 pg/ml in stimulated samples (Wilcoxon, p<0.01) from 10 volunteers (FIG. 9B). Background subtracted “CXCL9 responses” were calculated as: Stimulated CXCL9—unstimulated control CXCL9 concentrations (Signal−Noise [S/N]). Median S−N CXCL9 responses were 1234 pg/ml before vs. 7320 pg/ml (Wilcoxon, p<0.01) after the third vaccine dose (FIG. 10).

FIG. 11 shows serial CXCL9 responses (S−N) to stimulation with S-protein (0.125 μg) and N-protein (0.125 μg) after the third mRNA-1273 vaccine dose in a healthy volunteer. S-protein stimulation resulted in CXCL9 responses of 1472 pg/ml 51 days prior and 794 pg/ml immediately prior to the third vaccine dose (green line). The CXCL9 response was unchanged on post-vaccine Days 1 (832 pg/ml) and 2 (474 pg/ml) but increased starting on Day 5 and peaked at 27754 pg/ml on Day 19. CXCL9 responses decreased thereafter to 19208, 16448, 12656, 12942, 17008, and 12536 pg/ml on Days 26, 28, 39, 108, 187, and 325, respectively. The CXCL9 response to N-protein remained unchanged overall at: 266, 312, 1786, 1620, and 1160 pg/ml on Days 9, 39, 108, 187, and 325, respectively. Unstimulated control CXCL9 levels also remained unchanged, starting at 296 pg/ml on Day 0, peaking at 830 pg/ml on Day 2, and decreasing to 354, 294, 280, 407, and 412 pg/ml on Days 5, 39, 108, 187, and 325, respectively.

These findings are consistent with a memory T cell response in an individual vaccinated with an mRNA vaccine encoding the S-protein but not previously infected (minimal N-protein response). Stimulation with higher S-protein concentrations (0.25 μg, 0.375 μg) resulted in higher CXCL9 responses. Because the linear range of the Ella CXCL9 cartridge is 19.9-30,400 pg/ml, we chose stimulation with 0.125 μg of S- and N-proteins as the primary measures. FIG. 11 shows serial anti-RBD IgG antibody levels after the third vaccine dose, measured using the Abbott Alinity platform (blue line, right axis). Anti-RBD IgG levels were 511 arbitrary units (AU)/ml on Day 0 prior to the third vaccine dose and remained unchanged on Days 1 and 2 at 480 and 466 AU/ml, respectively. These levels dramatically increased starting Day 5 from 3849 to 57141, 42328, and 22739 AU/ml on Days 12, 39, and 108, respectively. Anti-N-protein levels measured qualitatively using the Alinity platform remained negative at all time points. This evaluation of healthy vaccine recipients shows the utility of the CRA in measuring antigen-specific T cell responses to SARS-COV-2 vaccination. It demonstrates a remarkable increase in both humoral and cellular immune responses for many but not all recipients after the third vaccine dose and suggests the durability of the T cell recall response at Day 325, as measured by the CRA.

### CXCL9 Responses Are Weak and Variable in Lung Transplant Recipients After Vaccination

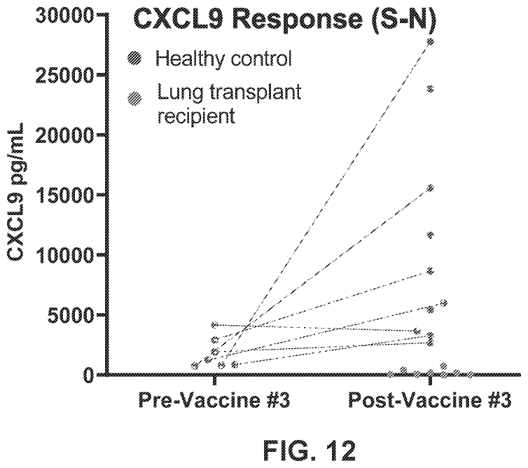

To evaluate CXCL9 responses among lung transplant recipients, we performed the CRA on whole-blood samples collected from eight lung transplant recipients at a median of 39 days after their third vaccine dose. The median CXCL9 response (S−N) to S-protein (0.125 μg) stimulation in transplant recipients (FIG. 12, red circles) after the third vaccine dose was 109 pg/ml vs. 7320 pg/ml in healthy controls (FIG. 12, blue circles; Wilcoxon, p<0.01).

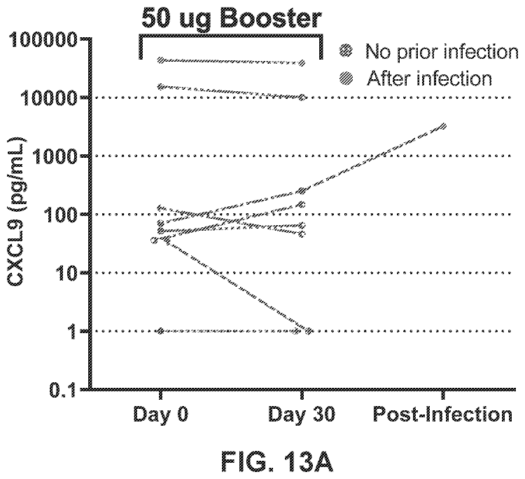

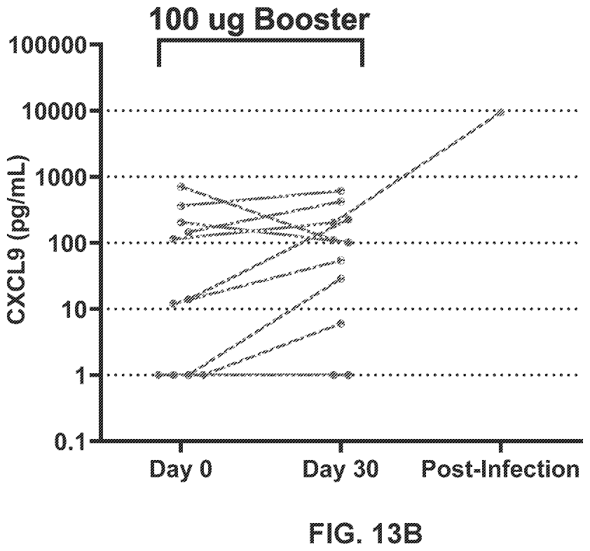

We recently completed enrollment in an investigator-initiated phase 1-2 trial in collaboration with Moderna to evaluate the safety and immunogenicity of higher-dose mRNA-1273 booster vaccines (fourth or fifth doses) in lung transplant patients. Sixty participants were scheduled to receive one of three booster vaccine doses, 50, 100, or 200 μg, with a dose escalation design. Unfortunately, trial enrollment was terminated early due to the availability of the bivalent mRNA-1273.222 booster. The following section describes the CXCL9 responses for the 19 patients enrolled in the 50-μg (n=8) and 100-μg (n=11) booster groups. Overall, both vaccine doses were well tolerated with minimal reactogenicity. CXCL9 responses were weak, with medians of only 61 and 106 pg/ml at Days 0 and 30, respectively, for the 50-μg booster group (Wilcoxon, p=0.18, FIG. 13A) and medians of 14 and 102 pg/ml at Days 0 and 30, respectively, for the 100-μg booster group (Wilcoxon, p=0.81, FIG. 13B). The 100-μg booster was not associated with higher Day 30 CXCL9 responses than the 50-μg booster (Wilcoxon, p=0.25). However, two participants had CXCL9 responses greater than 10000 pg/ml on Days 0 and 30, including one participant (red circle) with a positive CXCL9 response to N-protein (3256 pg/ml), likely indicating a prior asymptomatic infection. Two participants developed symptomatic SARS-COV-2 infections after Day 30, with corresponding increases in CXCL9 responses. One participant (FIG. 13B) had a CXCL9 response of 202 pg/ml on Day 30, SARS-COV-2 infection on Day 67, and a CXCL9 response of 9386 pg/ml 191 days after infection. The second participant (FIG. 13A) had a CXCL9 response of 252 pg/ml on Day 30, SARS-COV-2 infection on Day 207, and a CXCL9 response of 3220 pg/ml 55 days after infection. These results suggest that although lung transplant recipients have weak overall responses to mRNA-1273 vaccination, more robust CXCL9 responses are possible, particularly after SARS-COV-2 infection.

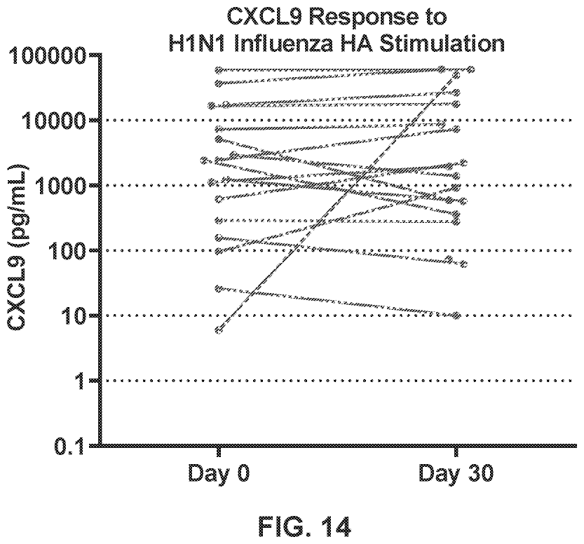

Compared with CXCL9 responses against SARS-COV-2 S-protein, the participants in the vaccine trial showed variable but overall stronger recall responses against the H1N1 influenza hemagglutinin (HA) peptide pool. Similar to the SARS-CoV-2 CRA protocol, 0.125 μg of H1N1 HA overlapping peptide pools (Peptivator, Miltenyi Biotec) were added to 0.8-ml whole-blood aliquots and incubated for 20 hours. The median CXCL9 response to HA stimulation was 1817 pg/ml on Day 0 and 1669 pg/ml on Day 30 (FIG. 14). 53% of participants had CXCL9 responses to HA stimulation greater than 1000 pg/ml on Day 30, whereas only 11% had a similar response to SARS-COV-2 S-protein stimulation. Stronger CXCL9 responses against influenza HA compared with SARS-Cov-2 S-protein may be due to several factors including pre-transplant and ongoing annual vaccination, vaccine type (inactivated virus vs. mRNA), high-dose influenza vaccine, and history of prior infections. FIG. 14 also shows the variability of CXCL9 responses against influenza HA peptides among participants, ranging from almost no response to above 60000 pg/ml. These results demonstrate that lung transplant recipients are able to mount robust (but variable) CXCL9 responses to viral peptide pool stimulation.

### The CRA Captures T Cell Responses After SARS-COV-2 Infection in Healthy Participants

FIG. 15 depicts CXCL9 responses and anti-RBD IgG levels in a previously healthy participant (#1) after SARS-COV-2 infection. This participant was vaccinated on Days—431 (first dose), —403 (second dose), and —116 (third dose). The CXCL9 response to S-protein increased as follows: 4674, 6668, 7120, 3774, and 16232 pg/ml on Days 3, 5, 6, 8 and 27, respectively (green line, left axis). The CXCL9 response to N-protein increased as follows: 60, 732, 2212, 4416, and 16440 pg/ml on Days 3, 5, 6, 8, and 27, respectively (brown line). Unstimulated control CXCL9 levels remained unchanged overall at 560, 514, 354, 358, and 304 pg/ml on Days 3, 5, 6, 8, and 27, respectively. The anti-RBD IgG level was 21897 AU/ml on Day 3 and remained stable at 27731 AU/ml on Day 27 (blue line, right axis). The anti-N-protein IgG was negative on Day 8 and positive on Day 27. This participant experienced rapid symptom resolution by Day 8.

A second participant (#2, FIG. 16) who was also healthy before SARS-COV-2 infection was previously vaccinated on Days-366,-338, and-51. The CXCL9 response to S-protein increased as follows: 5300, 5676, 5770, 6270, 9988, 10213, 14680, 26386, and 40774 pg/ml on Days 2, 3, 4, 6, 8, 16, 30, 68, and 149 respectively (green line). Compared with Participant #1, CXCL9 responses to N-protein occurred later and were smaller in magnitude: 0, 14, 0, 532, 9512, 8547, 6862, 2296, and 6771 pg/ml on Days 2, 3, 4, 6, 8, 16, 30, 68, and 149, respectively (brown line). Unstimulated control CXCL9 levels remained unchanged overall at 546, 388, 398, 328, 318, 149, 212, 326, and 321 pg/ml on Days 2, 3, 4, 6, 8, 16, 30, 68, and 149, respectively (not shown).

Anti-RBD IgG levels increased as follows: 10762, 11303, 10959, 13365, 18957, 21330, 29785, and 41117 AU/ml on Days 2, 3, 4, 6, 8, 11, 16, and 30, respectively (blue line). Anti-N-protein IgG first became positive on Day 11 and remained positive thereafter. Participant #2 developed mild to moderate long-COVID symptoms, including prolonged fatigue, autonomic dysfunction, and “brain fog” at 4 months post-infection.

Our disclosure demonstrates the ability of the CRA to quantify vaccine-induced T cell recall responses. The CRA offers several advantages over ELISpot and ICS, including (1) simplicity, (2) improved sensitivity, (3) the ability to evaluate the concurrent expression of hundreds of cytokines, and (4) the ability to determine the magnitude of cytokine release. Although ELISpot and ICS can capture the frequency of cytokine-specific responding cells (including polyfunctional cells), the CRA can measure the magnitude of the response (in pg/ml) for hundreds of cytokines concurrently, allowing for a detailed evaluation of cytokines and cytokine pathways involved in the recall response and their relative contributions (e.g., Th1:Th2 ratios, Th1:Th17 ratios). Embodiments of the invention can leverage the CRA to identify the key cytokine and cytokine pathways involved in the recall response to SARS-COV-2 vaccination, and use this understanding to develop the CRA as a reliable, easy to measure and rapidly deployable assay that quantifies an individual's cellular immunity against SARS-COV-2. By adjusting the stimulating peptides, the CRA can be rapidly updated to quantify vaccine-induced T cell immunity against new variants. The CRA is easily standardized across laboratories and can allow the assay to be performed rigorously at individual clinical trial sites in a decentralized manner. Simplicity, low cost, and the production of abundant data make the CRA an efficient and effective method for studying vaccine-induced T cell recall responses in large numbers of participants.

Embodiments of the invention leverage our novel whole-blood T cell stimulation assay (CRA) for quantifying T cell recall responses to SARS-COV-2 vaccination. We can leverage the CRA to delineate the cytokines and cytokine pathways involved in the recall response and identify the key cytokine responses to be used as a cellular mechanistic correlate of protection against SARS-COV-2. The CRA can improve our assessment of the cytokine interactions involved in the T cell recall response, and provide a simple, inexpensive T cell recall assay to support vaccine development and optimize vaccine dosing by allowing the identification of individuals with inadequate cellular immunity.

Embodiments of the invention can be used to improve the understanding of the cytokine responses (Th1, Th2, Th17, Treg) involved in the SARS-COV-2 vaccine induced T cell recall response, as well as the cellular sources of the cytokines. Embodiments of the invention can be used for the determination of the correlation between CRA and ICS responses for all key cytokines identified. Embodiments of the invention can be used for the determination of the levels of the CRA response that protect against severe disease in a cohort of high-risk individuals including lung transplant recipients. Embodiments of the invention can include the use of SARS-COV-2 overlapping peptide pools for S-protein (WA1/2020, BA.4/5), N-protein (BA.4/5), 2.5 μg PHA (positive control) in single use aliquots, 5 ml sterile culture tubes, Ella machine and cartridges for the key cytokines identified. Stimulating peptides can be adjusted to correspond to any changes in SARS-COV-2 vaccine formulations and novel SARS-COV-2 variants identified.

Cytokines measurement can be performed on Ella, a commercially available, well-validated and fully automated microfluidic ELISA platform able to run 72 samples on a single cartridge in 80 mins with no additional labor required. The platform uses pneumatic pistons and values to direct the sample through microfluidic channels into three “glass nanoreactors”, where three sandwich ELISAs are captured for each sample. Thus, each analyte is detected in triplicate, improving both precision and sensitivity compared with standard ELISA platforms. Ella is a robust platform with over 200 analytes validated on a research use only basis. There are numerous cartridge formats available in terms of sample size (16, 32, 72-sample) and channel number (up to 8) allowing for multiplex testing. The microfluidic channels are designed in parallel eliminating potential cross reactivity (e.g., CXCL9 antibodies remain in the CXCL9 channel only). Thus, the Ella platform can accurately detect up to 8 cytokines from a single sample in a rapid and fully-automated manner. These qualities make the CRA easy to scale-up and standardize across laboratories allowing for decentralized sample analysis in clinical trials.

This Example describes a novel assay to quantify SARS-COV-2 specific T cell responses as a mechanistic correlate of protection against SARS-COV-2. The CRA has several advantages over ICS and ELISpot including simplicity, improved sensitivity and the ability to evaluate the concurrent expression of hundreds of cytokines. Embodiments of the invention can leverage the CRA to identify the key cytokine and cytokine pathways involved in the SARS-COV-2 vaccine-induced recall response, and use this understanding to develop the CRA as a sensitive and reliable assay of SARS-CoV-2 cellular immunity. By adjusting the stimulating SARS-COV-2 peptide pools, the CRA can be rapidly updated to quantify cellular responses for any new vaccine or novel SARS-COV-2 variant. The CRA is easily scaled-up and standardized across laboratories, allowing for decentralized sample analysis in clinical trials. Simplicity, low cost, and the production of abundant data make the CRA an efficient and effective method for studying vaccine-induced T cell recall responses in large numbers of participants.

The CRA has an important advantage over humoral assays (NAbs and binding antibodies) in its ability to quantify immune responses against internal viral proteins. This can allow the CRA to be used to evaluate next generation vaccines involving conserved internal components of the SARS-COV-2 virus (e.g., N-protein, ORFs, etc.), a clear advantage over humoral assays that measure binding of conformational epitopes on the SARS-COV-2 surface. Furthermore, there is increasing evidence that while NAbs mediate the rapidly waning protection against infection (due to RBD evolution of the variants), cellular responses mediate the durable protection from severe disease that is the primary goal of the SARS-COV-2 vaccines.

Embodiments of the invention can improve our understanding of the SARS-CoV-2 mRNA vaccine-induced T cell recall response and provide a sensitive and reliable assay of cellular immunity to support vaccine development and evaluate vaccine efficacy against novel variants. We can also determine the levels of the CRA responses that protect against severe disease in a cohort of high-risk individuals including lung transplant recipients. Thus, the CRA may become a valuable tool for identifying individuals with inadequate cellular immune responses who would benefit from additional (higher dose or more frequent) booster doses, as a strategy to improve outcomes in high-risk groups who continue to have poor outcomes after SARS-COV-2 infection.

### EXAMPLE 1 REFERENCES

1. Baden LR, El Sahly HM, Essink B, et al. Efficacy and Safety of the mRNA-1273 SARS-COV-2 Vaccine. N Engl J Med. 2021; 384(5):403-416. doi:10.1056/nejmoa2035389
2. Khoury DS, Cromer D, Reynaldi A, et al. Neutralizing antibody levels are highly predictive of immune protection from symptomatic SARS-COV-2 infection. Nat Med. 2021; 27(7):1205-1211. doi:10.1038/s41591-021-01377-8
3. Gilbert PB, Montefiori DC, McDermott AB, et al. Immune correlates analysis of the mRNA-1273 COVID-19 vaccine efficacy clinical trial. Science (80-). 2022; 375(6576):43-50. doi:10.1126/science.abm3425
4. Feng S, Phillips DJ, White T, et al. Correlates of protection against symptomatic and asymptomatic SARS-COV-2 infection. Nat Med. 2021; 27(11):2032-2040. doi:10.1038/s41591-021-01540-1
5. Keeton R, Tincho MB, Ngomti A, et al. T cell responses to SARS-COV-2 spike cross-recognize Omicron. Nature. Published online 2022:1-5. doi:10.1038/s41586-022-04460-3
6. Sekine T, Perez-Potti A, Rivera-Ballesteros O, et al. Robust T Cell Immunity in Convalescent Individuals with Asymptomatic or Mild COVID-19. Cell. 2020; 183(1):158-168.e14. doi:10.1016/j.cell.2020.08.017
7. Zuo J, Dowell AC, Pearce H, et al. Robust SARS-COV-2-specific T cell immunity is maintained at 6 months following primary infection. Nat Immunol. 2021; 22(5):620-626. doi:10.1038/s41590-021-00902-8
8. Lyke KE, Atmar RL, Islas CD, et al. Rapid decline in vaccine-boosted neutralizing antibodies against SARS-COV-2 Omicron variant. Cell Reports Med. 2022; 3(7). doi:10.1016/j.xcrm.2022.100679
9. Lassaunière R, Polacek C, Frische A, et al. Neutralizing Antibodies Against the SARS-COV-2 Omicron Variant (BA.1) 1 to 18 Weeks after the Second and Third Doses of the BNT162b2 mRNA Vaccine. JAMA Netw Open. 2022; 5(5):E2212073. doi:10.1001/jamanetworkopen.2022.12073
10. Zhang GF, Meng W, Chen L, et al. Journal of Medical Virology—2022—Zhang—Neutralizing antibodies to SARS-COV-2 variants of concern including Delta and Omicron.pdf.
11. Evans JP, Zeng C, Carlin C, et al. Neutralizing antibody responses elicited by SARS-COV-2 mRNA vaccination wane over time and are boosted by breakthrough infection. Sci Transl Med. 2022; 14(637). doi:10.1126/scitranslmed.abn8057
12. Geers D, Shamier MC, Bogers S, et al. SARS-COV-2 variants of concern partially escape humoral but not T-cell responses in COVID-19 convalescent donors and vaccinees. Sci Immunol. 2021; 6(59):1-15. doi:10.1126/sciimmunol.abj1750
13. Goel RR, Painter MM, Apostolidis SA, et al. mRNA vaccines induce durable immune memory to SARS-COV-2 and variants of concern. 2021; 0829. doi:10.1126/science.abm0829
14. Zhang Z, Mateus J, Coelho CH, et al. Humoral and cellular immune memory to four COVID-19 vaccines. Cell. 2022; 185(14):2434-2451.e17. doi:10.1016/j.cell.2022.05.022
15. Tarke A, Sidney J, Methot N, et al. Impact of SARS-COV-2 variants on the total CD4+and CD8+ T cell reactivity in infected or vaccinated individuals. Cell Reports Med. 2021; 2(7):100355. doi:10.1016/j.xcrm.2021.100355
16. Ssentongo P, Ssentongo A E, Voleti N, et al. SARS-COV-2 vaccine effectiveness against infection, symptomatic and severe COVID-19: a systematic review and meta-analysis. BMC Infect Dis. 2022; 22(1):1-12. doi:10.1186/s12879-022-07418-y
17. Külper-Schiek W, Piechotta V, Pilic A, et al. Facing the Omicron variant—how well do vaccines protect against mild and severe COVID-19? Third interim analysis of a living systematic review. Front Immunol. 2022; 13(August):1-13. doi:10.3389/fimmu.2022.940562
18. Tseng HF, Ackerson BK, Luo Y, et al. Effectiveness of mRNA-1273 against SARS-COV-2 Omicron and Delta variants. Nat Med. 2022; 28(5):1063-1071. doi:10.1038/s41591-022-01753-y
19. Andrews N, Tessier E, Stowe J, et al. Duration of Protection against Mild and Severe Disease by Covid-19 Vaccines. N Engl J Med. 2022; 386(4):340-350. doi:10.1056/nejmoa2115481
20. Barda N, Dagan N, Cohen C, et al. Effectiveness of a third dose of the BNT162b2 mRNA COVID-19 vaccine for preventing severe outcomes in Israel: an observational study. Lancet. 2021; 398(10316):2093-2100. doi:10.1016/S0140-6736(21) 02249-2
21. Tan AT, Linster M, Tan CW, et al. Early induction of functional SARS-COV-2-specific T cells associates with rapid viral clearance and mild disease in COVID-19 patients. Cell Rep. 2021; 34(6):108728. doi:10.1016/j.celrep.2021.108728
22. Bange EM, Han NA, Wileyto P, et al. CD8+ T cells contribute to survival in patients with COVID-19 and hematologic cancer. Nat Med. 2021; 27(7):1280-1289. doi:10.1038/s41591-021-01386-7
23. McMahan K, Yu J, Mercado NB, et al. Correlates of protection against SARS-CoV-2 in rhesus macaques. Nature. 2021; 590(7847):630-634. doi:10.1038/s41586-020-03041-6
24 Jiang X, Clark RA, Liu L, Wagers AJ, Fuhlbrigge RC, Kupper TS. Skin infection generates non-migratory memory CD8 +T RM cells providing global skin immunity. Nature. 2012; 483(7388):227-231. doi:10.1038/nature10851
25. Schenkel JM, Fraser KA, Vezys V, Masopust D. Sensing and alarm function of resident memory CD8 +T cells. Nat Immunol. 2013; 14(5):509-513. doi:10.1038/ni.2568
26. Iijima N, Iwasaki A. A local macrophage chemokine network sustains protective tissue-resident memory CD4 T cells. Science (80-). 2014; 346(6205):93-98. doi:10.1126/science. 1257530
27. Andrews N, Stowe J, Kirsebom F, et al. Covid-19 Vaccine Effectiveness against the Omicron (B.1.1.529) Variant. N Engl J Med. 2022; 386(16):1532-1546. doi:10.1056/nejmoa2119451
28. Liu L, Iketani S, Guo Y, et al. Striking antibody evasion manifested by the Omicron variant of SARS-COV-2. Nature. 2022; 602(7898):676-681. doi:10.1038/s41586-021-04388-0
29. Hoffmann M, Krüger N, Schulz S, et al. The Omicron variant is highly resistant against antibody-mediated neutralization: Implications for control of the COVID-19 pandemic. Cell. 2022; 185(3):447-456.e11. doi:10.1016/j.cell.2021.12.032
30 Planas D, Saunders N, Maes P, et al. Considerable escape of SARS-COV-2 Omicron to antibody neutralization. Nature. 2022; 602(7898):671-675. doi:10.1038/s41586-021-04389-z
31. Liu J, Chandrashekar A, Sellers D, et al. Vaccines elicit highly conserved cellular immunity to SARS-COV-2 Omicron. Nature. 2022; 603(7901):493-496. doi:10.1038/s41586-022-04465-y
32. Tarke A, Coelho CH, Zhang Z, et al. SARS-COV-2 vaccination induces immunological T cell memory able to cross-recognize variants from Alpha to Omicron. Cell. 2022; 185(5):847-859.e11. doi:10.1016/j.cell.2022.01.015
33. Jordan SC, Shin BH, Gadsden TAM, et al. T cell immune responses to SARS-CoV-2 and variants of concern (Alpha and Delta) in infected and vaccinated individuals. Cell Mol Immunol. 2021; 18(11):2554-2556. doi:10.1038/s41423-021-00767-9
34. De Marco L, D′Orso S, Pirronello M, et al. Assessment of T-cell Reactivity to the SARS-COV-2 Omicron Variant by Immunized Individuals. JAMA Netw Open. 2022; 5(4):E2210871. doi:10.1001/jamanetworkopen.2022.10871
35. Boyarsky BJ, Werbel WA, Avery RK, et al. Antibody response to 2-dose sars-cov-2 mrna vaccine series in solid organ transplant recipients. JAMA—J Am Med Assoc. 2021; 325(21):2204-2206. doi:10.1001/jama.2021.7489
36. Hall VG, Ferreira VH, Ierullo M, et al. Humoral and cellular immune response and safety of two-dose SARS-COV-2 mRNA-1273 vaccine in solid organ transplant recipients. Am J Transplant. 2021; 21(12):3980-3989. doi:10.1111/ajt.16766
37. Marion O, Del Bello A, Bravanel F. Safety and Immunogenicity of Anti-SARS-COV-2 Messenger RNA Vaccines in Recipients of Solid Organ Transplants. Ann Intern Med. 2021; 174(9):1336-1338.
38. Schramm R, Costard-Jäckle A, Rivinius R, et al. Poor humoral and T-cell response to two-dose SARS-COV-2 messenger RNA vaccine BNT162b2 in cardiothoracic transplant recipients. Clin Res Cardiol. 2021; 110(8):1142-1149. doi:10.1007/s00392-021-01880-5
39. Shostak Y, Shafran N, Heching M, et al. Early humoral response among lung transplant recipients vaccinated with BNT162b2 vaccine. Lancet Respir Med. 2021; 9(6):e52-e53. doi:10.1016/S2213-2600(21) 00184-3
40. Havlin J, Svorcova M, Dvorackova E, et al. Immunogenicity of BNT162b2 mRNA COVID-19 vaccine and SARS-COV-2 infection in lung transplant recipients. J Hear Lung Transplant. 2021; 40(8):754-758. doi:10.1016/j.healun.2021.05.004
41. Ibarrondo FJ, Anton PA, Fuerst M, et al. Parallel Human Immunodeficiency Virus Type 1-Specific CD8 +T-Lymphocyte Responses in Blood and Mucosa during Chronic Infection. J Virol. 2005; 79(7):4289-4297. doi:10.1128/jvi.79.7.4289-4297.2005
42. Taus E, Hofmann C, Ibarrondo FJ, et al. Dominant CD8+ T Cell Nucleocapsid Targeting in SARS-COV-2 Infection and Broad Spike Targeting From Vaccination. Front Immunol. 2022; 13(February):1-12. doi:10.3389/fimmu.2022.835830
43. Bihl FK, Loggi E, Chisholm J V., et al. Simultaneous assessment of cytotoxic T lymphocyte responses against multiple viral infections by combined usage of optimal epitope matrices, anti-CD3 mAb T-cell expansion and “RecycleSpot.” J Transl Med. 2005; 3(June). doi:10.1186/1479-5876-3-20
44. Jones N, Agrawal D, Elrefaei M, et al. Evaluation of antigen-specific responses using in vitro enriched T cells. J Immunol Methods. 2003; 274(1-2):139-147. doi:10.1016/S0022-1759(02) 00510-0
45. Taus E, Hofmann C, Ibarrondo F, et al. Persistent Memory Despite Rapid Contraction of Circulating T Cell Responses to SARS-COV-2 mRNA Vaccination. Front Immunol. 2023; Accepted f.
46. Lustig Y, Gonen T, Meltzer L, et al. Superior immunogenicity and effectiveness of the third compared to the second BNT162b2 vaccine dose. Nat Immunol. 2022; 23(6):940-946. doi:10.1038/s41590-022-01212-3
47. Saciuk Y, Kertes J, Shamir Stein N, Ekka Zohar A. Effectiveness of a Third Dose of BNT162b2 mRNA Vaccine. J Infect Dis. 2022; 225(1):30-33. doi:10.1093/infdis/jiab556
48. Bar-On YM, Goldberg Y, Mandel M, et al. Protection of BNT162b2 Vaccine Booster against Covid-19 in Israel. N Engl J Med. 2021; 385(15):1393-1400.doi:10.1056/nejmoa2114255
49. Griffith JW, Sokol CL, Luster AD. Chemokines and chemokine receptors: Positioning cells for host defense and immunity. Annu Rev Immunol. 2014; 32:659-702. doi:10.1146/annurev-immunol-032713-120145
50. Kaech SM, Wherry EJ, Ahmed R. Effector and memory T-cell differentiation: Implications for vaccine development. Nat Rev Immunol. 2002; 2(4):251-262. doi:10.1038/nri778
51. Mescher MF, Curtsinger JM, Agarwal P, et al. Signals required for programming effector and memory development by CD8+ T cells. Immunol Rev. 2006; 211:81-92. doi:10.1111/j.0105-2896.2006.00382.x
52. Curtsinger JM, Valenzuela JO, Agarwal P, Lins D, Mescher MF. Cutting Edge: Type I IFNs Provide a Third Signal to CD8 T Cells to Stimulate Clonal Expansion and Differentiation. J Immunol. 2005; 174(8):4465-4469. doi:10.4049/jimmunol. 174.8.4465
53. Rahimi RA, Luster AD. Chemokines: Critical Regulators of Memory T Cell Development, Maintenance, and Function. Vol 138. 1st ed. Elsevier Inc.; 2018. doi:10.1016/bs.ai.2018.02.002
54. Glennie ND, Yeramilli VA, Beiting DP, Volk SW, Weaver CT, Scott P. Skin-resident memory CD4+T cells enhance protection against Leishmania major infection. J Exp Med. 2015; 212(9):1405-1414. doi:10.1084/jem.20142101
55. Shino M, DerHovanessian A, Sayah D, et al. The Impact of Allograft CXCL9 during Respiratory Infection on the Risk of Chronic Lung Allograft Dysfunction. OBM Transpl. 2018; 2(4).
56. Shino MY, Li N, Todd JL, et al. Correlation between BAL CXCR3 chemokines and lung allograft histopathologies: A multicenter study. Am J Transplant. Published online 2021. doi:10.1111/ajt.16601
57. Shino MY, Weigt SS, Li N, et al. The Prognostic Importance of Bronchoalveolar Lavage Fluid CXCL9 During Minimal Acute Rejection on the Risk of Chronic Lung Allograft Dysfunction. Am J Transplant. 2018; 18(1):136-144. doi:10.1111/ajt.14397
58. Shino MY, Zhang Q, Li N, et al. The allograft injury marker CXCL9determines prognosis of anti-HLA antibodies after lung transplantation. Am J Transplant. 2022; 22(2):565-573. doi:10.1111/ajt.16827

### Example 2: Observing CXCR3 Ligand Responses In Allo-Specific Lymphocyte Stimulation

Embodiments of the invention can be used to observe CXCR3 ligand responses in allo-specific lymphocyte stimulation.

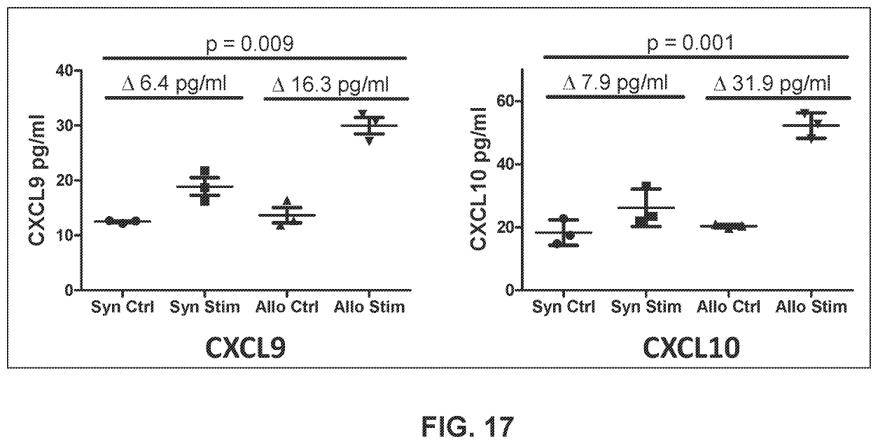

Murine Tracheal Transplant Model: We hypothesized that one of the key events triggering the deleterious cycle of CXCR3 induced mononuclear cell recruitment and cell damage after allogenic organ transplantation is the allo-specific stimulation of memory T-cells by antigen presenting cells (FIG. 5). Thus, we sought to further study CXCR3 ligand responses after allo-specific memory T-cell stimulation in a novel lymphocyte recall assay. A murine heterotopic tracheal transplant model previously established by our group was used. Donor tracheas were transplanted subcutaneously into syngeneic control (“Syn”:C57BL/6 to C57BL/6) and allogeneic mice (“Allo”: BALB/c to C57BL/6). After a 6-month period of allo-sensitization, recipient splenocytes were harvested. Previously frozen splenocytes from the trachea donor were thawed and irradiated at 3000 rad to prevent chemokine release by these cells. Using appropriate negative controls, 300,000 recipient splenocytes were stimulated with 300,000 irradiated donor cells in a RPMI with FBS media. After 18 hours of stimulation, IFN-γ, CXCL9 and CXCL10 concentrations in the supernatant were measured by luminex. Irradiation effectively eliminated chemokine release from donor splenocytes: mean CXCL9 and CXCL10 concentrations for irradiated donor splenocytes after 18 hours of culture in the same media were 7.3 and 4.4 pg/ml, respectively. Recipient splenocytes stimulated with donor splenocytes (“Syn Stim” and “Allo Stim”) showed higher CXCL9 and CXCL10 concentrations compared with unstimulated recipient splenocytes (“Syn Ctrl” and “Allo Ctrl”, FIG. 17). The increase in chemokine concentrations after stimulation with donor splenocytes was significantly higher for the allogeneic transplants compared with the syngeneic controls. The increase in CXCL9 concentration was 16.3 vs 6.4 pg/ml (p=0.009) for the allogeneic and syngeneic transplants, respectively. The increase in CXCL10 concentration was 31.9 vs 7.9 pg/ml (p=0.001) for the allogeneic and syngeneic transplants. Since only memory T-cells primed against a specific antigen are able to mount an immune response within the first 24 hours, we speculate that allo-sensitized memory T-cells are responsible for this immune response. Of note, IFN-γ responses were weak, below our level of detection for all control and stimulated samples.

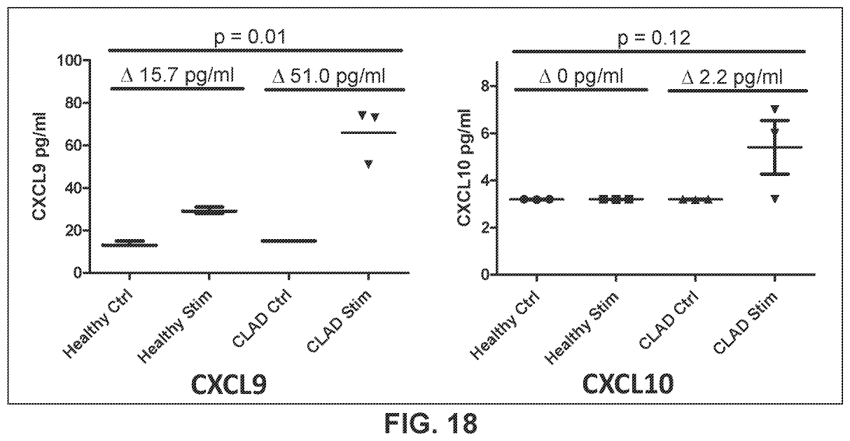

Human Lung Transplant Recipients: We next explored CXCR3 ligand responses after allospecific lymphocyte stimulation in human lung transplant recipients. Peripheral blood mononuclear cells (PBMCs) from two transplant recipients, both approximately 3-years posttransplant, were obtained by blood draw. One patient was “healthy” with good lung function, while the other was diagnosed with CLAD 3 months earlier “CLAD”. Previously frozen donor lung lymph node cells for each recipient were thawed and irradiated to prevent chemokine release. Recipient PBMCs were stimulated with donor lymph node cells for 18 hours, and IFN-γ/CXCR3 chemokine concentrations in the supernatant were measured by luminex. Recipient PBMCs stimulated with donor cells (“Healthy Stim” and “CLAD Stim”) produced higher CXCL9 concentrations in the supernatant compared with unstimulated PMBCs (“Healthy Ctrl” and “CLAD Ctrl”, FIG. 18). The increase in CXCL9 concentration after stimulation with donor cells was significantly higher for the “CLAD” recipient compared with the “healthy”. The increase in CXCL9 was 51.0 vs 15.7 pg/ml (p=0.01) for the CLAD and healthy recipients, respectively. The increase in CXCL10 concentrations after donor cell stimulation was also higher for the “CLAD” recipient compared with the “healthy”, but the trend was not statistically significant. Of note, IFN-γ responses were weak, near the lower limit of quantification (LLOQ) for both stimulated and unstimulated samples. These data support our hypothesis that CXCR3 ligands are key mediators of the immune response after allo-specific memory T-cell stimulation, and that the strength of this immune response may be quantified by CXCR3 ligand concentrations.

Embodiments of our assays quantify CXCR3 ligands (as well as other cytokines/chemokines) instead of IFN-γ in order to take advantage of signal amplification to improve the sensitivity of the lymphocyte stimulation (recall) assay. We can use a number of lymphocyte stimulators including but not limited to donor cells/tissue, peptides from infectious microorganisms, vaccines and proteins derived from vaccinations, tumor/malignant cells, as well as other proteins of interest, including non-specific lymphocyte stimulators (PMA, PHA, LPS etc.). We can quantify CXCR3 ligand responses in various ways including but not limited to supernatant concentrations, plasma concentrations, CXCR3 ligand ELISPOTs, CXCR3 ligand intracellular staining and PCR. This novel method of assessing the immune response after transplantation may allow for improved immune monitoring and tailoring of immunosuppressive medications, as a way to improve post-transplant outcomes. It may also be useful in non-transplant/healthy patients as way to evaluate immune responses including post-vaccination/post-infection immune responses, anti-tumor/anti-malignancy immune responses and post-immunomodulatory treatment immune responses.

Methods, materials and systems that can be adapted for performing aspects of the claims invention are disclosed in U.S. Patent Application Publication Nos.: 20200291480, 20200277643, 20200200707, 20200155966, 20200062768, 20190369068, 20190369048, 20190285634, 20190257827, 20190248885, 20190218531, 20180321189, 20170275700, 20170151564, 20170065978, 20170050186, 20170001197, 20160370319, 20160230203, 20150241389, 20140106372, 20130167937, and 20120213667; as well as PCT Application Serial No. PCT/US21/59934, and Oliveira et al., Rev Inst Med Trop Sao Paulo 2020 Jun. 29; 62:e44. doi:10.1590/S1678-9946202062044; Kohmer et al., J Clin Virol. 2020 August; 129:104480. doi: 10.1016/j.jcv.2020.104480. Epub 2020; and Wang et al., Emerg

Microbes Infect. 2020 December; 9(1):2200-2211. doi: 10.1080/22221751.2020.1826362; the contents of all of which are incorporated herein by reference.

All publications mentioned herein are incorporated by reference to disclose and describe aspects, methods and/or materials in connection with the cited publications. Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

Claims:

1. A method of observing an immune response to an antigen, the method comprising:
obtaining immune cells from the whole blood of a subject;
incubating the immune cells with the antigen;
observing concentrations of one or more cytokines released from the immune cells in response to incubation with the antigen;
comparing concentrations of the one or more cytokines released from the immune cells in response to incubation with the antigen to control immune cells, wherein the control immune cells comprise immune cells from the whole blood of the subject that are not incubated with antigen; and
observing the presence or absence of an immune response, wherein an immune response is observed when concentrations of the one or more cytokines released from the immune cells in response to incubation with the antigen are at least two-fold greater than concentrations of the one or more cytokines released from control immune cells.

2. The method of claim 1, wherein the one or more cytokines includes at least one of CXCL9, CXCL10, interferon-γ, IL-2, tumor necrosis factor α, IL-18, IL-4, IL-5, IL-9, IL-13, IL-1, IL-6, IL-17, IL12, CCL20, IL10, IL-35, and tumor growth factor β.

3. The method of claim 1, wherein the one or more cytokines include at least two or three different cytokines.

4. The method of claim 1, wherein the one or more cytokines are measured in whole-blood, plasma, serum or culture media/supernatant compartments.

5. The method of claim 1, wherein the antigen comprises a polypeptide.

6. The method of claim 1, wherein the antigen comprises pooled overlapping peptides spanning a protein.

7. The method of claim 1, wherein the antigen comprises an antigen found in a virus, bacteria, fungus or mycobacterium.

8. The method of claim 1, wherein the antigen comprises a malignant cell.

9. The method of claim 1, wherein the antigen comprises a cell from an organ transplant donor.

10. The method of claim 1, wherein the antigen comprises HLA molecules.

11. The method of claim 1, wherein the antigen comprises non-specific lymphocyte stimulators including, but not limited to phytohemagglutinin [PHA], phorbaol 12-myristate 13-acetate [PMA], lysophosphatidylcholine [LPS]).

12. The method of claim 1, wherein immune cells are incubated with the antigen for at least 10 or 24 hours.

13. The method of claim 1, wherein observing concentrations of one or more cytokines released from the immune cells includes binding a cytokine with a capture antibody in an assay device comprising microfluidic channels and the capture antibody is coupled to one or more regions within the microfluidic channels.

14. The method of claim 1, wherein the subject is selected to be:
a patient immunized with a vaccine;
a patient who has undergone a cell or tissue transplantation procedure;
a patient diagnosed as having an infectious disease;
a patient diagnosed as having an immune disorder;
a patient administered an immunomodulatory agent;
a patient diagnosed with a malignancy.

15. A system for observing immune response in an individual, the system comprising at least two of:
(a) an antibody that binds to a CXCL9 polypeptide and an agent selected for its ability to image the antibody bound to the CXCL9 polypeptide;
(b) an antibody that binds to a CXCL10 polypeptide and an agent selected for its ability to image the antibody bound to the CXCL10 polypeptide; and
(c) an antibody that binds to an IFN-γ polypeptide and an agent selected for its ability to image the antibody bound to the IFN-γ polypeptide.

16. The system of claim 15, wherein the system comprises at least one of:
(a) a detection antibody that binds to a CXCL9 polypeptide, wherein the detection antibody is coupled to a detectable label; and a capture antibody that binds to a CXCL9 polypeptide wherein the capture antibody is coupled to a matrix;
(b) a detection antibody that binds to a CXCL10 polypeptide, wherein the detection antibody is coupled to a detectable label; and a capture antibody that binds to a CXCL10 polypeptide wherein the capture antibody is coupled to a matrix; and
(c) a detection antibody that binds to an IFN-γ polypeptide, wherein the detection antibody is coupled to a detectable label; and a capture antibody that binds to a IFN-γ polypeptide wherein the capture antibody is coupled to a matrix.

17. The system of claim 16, wherein the system comprises (a)-(c).

18. The system of claim 16, wherein the system comprises microfluidic channels and the detection antibody is coupled to one or more regions in the microfluidic channels.

19. The system of claim 18, wherein fluid test samples and reagents are directed by pneumatic pistons and valves through the microfluidic channels wherein the microfluidic channels are configured to perform at least three sandwich ELISAs.

20. A method of observing an immune response comprising:
obtaining a fluid sample selected to include at least one of CXCL9, CXCL10 and IFN-γ, IL-2, tumor necrosis factor «, IL-18, IL-4, IL-5, IL-9, IL-13, IL-1, IL-6, IL-17, IL12, CCL20, IL10, IL-35, tumor growth factor β, wherein the CXCL9, CXCL10 and IFN-γ were generated by immune cells as part of an immune response;
disposing the fluid sample in the system of claim 15 so that concentrations of the at least one of CXCL9, CXCL10, IFN-γ, IL-2, tumor necrosis factor α, IL-18, IL-4, IL-5, IL-9, IL-13, IL-1, IL-6, IL-17, IL12, CCL20, IL10, IL-35, and tumor growth factor β, are observed; and
correlating concentrations of at least one of CXCL9, CXCL10, IFN-γ, IL-2, tumor necrosis factor α, IL-18, IL-4, IL-5, IL-9, IL-13, IL-1, IL-6, IL-17, IL12, CCL20, IL10, IL-35, tumor growth factor β with an immune response, such that an immune response is observed.

21. The method of claim 15, wherein the immune response comprises an immune response to a vaccination.

22. The method of claim 15, wherein the immune response comprises an immune response to transplanted tissues.

23. The method of claim 15, wherein the immune response comprises an immune response to an infectious agent.

24. The method of claim 15, wherein the immune cells are obtained from a subject administered a therapeutic agent or an immunomodulatory agent.
